(19) United States
(12) Patent Application Publication
(10) Pub. No.: US 2004/0258669 A1
(43) Pub. Date: Dec. 23, 2004

(54) Mesenchymal stem cells and methods of use thereof

(76) Inventors: Victor J. Dzau, Newton, MA (US); Abeel Mangi, Brookline, MA (US); James Edmund Ip, Cambridge, MA (US)

Correspondence Address: Ingrid A. Beattie, Ph.D., J.D., Mintz, Levin, Cohn, Ferris,, Glovsky and Popeo, P.C., One Financial Center, Boston, MA, 02111, US

(21) Appl. No.: 10/701,789

(22) Filed: Nov. 5, 2003

Related U.S. Application Data
(60) Provisional application No. 60/423,805, filed on Nov. 5, 2002.
(60) Provisional application No. 60/493,874, filed on Aug. 8, 2003.

(51) Int. Cl. A61K 48/00; C12N 5/08

(52) U.S. Cl. 424/93.21; 435/366; 435/368

(57) ABSTRACT

The invention provides compositions and methods of enhancing the viability of primary stem cells and enhancing the engraftment of transplanted stem cells into a mammalian recipient. Accordingly, the invention includes a method of regenerating a mesenchymally-derived tissue by contacting the tissue with a composition containing an isolated adult mesenchymal stem cell, which are apoptosis-resistant. The mesenchymal stem cell is an adult cell obtained from an adult bone marrow.

### RELATED APPLICATIONS

This application claims the benefit of U.S. Ser. No. 60/423,805, filed Nov. 5, 2002; and U.S. Ser. No. 60/493,874, filed Aug. 8, 2003, which are incorporated herein by reference in their entirety.

### STATEMENT AS TO FEDERALLY SPONSORED RESEARCH

This invention was made with U.S. government support under National Institutes of Health grants. The government has certain rights in the invention.

### FIELD OF THE INVENTION

The invention relates to modified mesenchymal stem cells and methods of treating injury or disease.

### BACKGROUND OF THE INVENTION

Patient mortality and morbidity is increased by cell/tissue damage or death resulting from acute and chronic injury or disease, such as myocardial infarction, cardiac failure, stroke, degenerative neurological disease, spinal injury, musculoskeletal diseases, hypertension, and diabetes. It is of great importance to determine methods by which new cells can prevent, reduce, and/or repair this damage.

### SUMMARY OF THE INVENTION

The invention provides compositions and methods of enhancing the viability of primary stem cells and enhancing the engraftment of transplanted stem cells into a mammalian recipient. Accordingly, the invention includes a method of regenerating a mesenchymally-derived tissue by contacting the tissue with a composition containing an isolated adult mesenchymal stem cell. The mesenchymal stem cell is an adult cell obtained from an adult bone marrow. The cell contains an exogenous nucleic acid encoding an akt gene. Preferably, the nucleic acid is introduced into the cell, e.g., transduced with a retroviral vector containing the gene, ex vivo. Following introduction of an akt gene into the cell, a population of recombinant stem cells is introduced or reintroduced, into a mammalian recipient.

A mesenchymally-derived tissue is one characterized by an embryonic origin in the mesoderm. The mesenchyme is a part of the mesoderm from which connective tissues, blood vessels, heart tissue, and lymphatic tissue is derived. Mesenchymal cells differentiate into connective, epithelial, nervous and muscle tissues. For example, the target tissue is selected from the group consisting of myocardial, brain, spinal cord, bone, cartilage, liver, muscle, lung, vascular, and adipose tissue, and the engrafted stem cells differentiate into the tissue type of the target tissue following engraftment. The muscle tissue is skeletal muscle or smooth muscle, e.g., vascular smooth muscle cells, and the method is used to regenerate muscle tissue in subjects suffering from or at risk of developing acute or chronic degenerative disease, e.g., muscular dystrophy such as Duchenne's muscular dystrophy. The epithelial tissue contains skin, intestinal, or other tissue-specific epithelial cells. Neuronal tissue includes brain, spinal cord tissue; the methods are useful in regenerating damaged neuronal tissue, e.g., brain tissue, following a stroke, or minimizing damage to neuronal tissue prior to a traumatic event such as surgery.

Migration of stem cells to target tissues is enhanced by further genetic modification, e.g., introduction of an exogenous nucleic acid encoding a homing molecule into the cells. Examples of homing molecules include chemokine receptors, interleukin receptors, estrogen receptors, and integrin receptors. The cells optionally contain an exogenous nucleic acid encoding a gene product, which increases endocrine action of the cell, e.g., a gene encoding a hormone, or a paracrine action of the cell. For example, stem cells are genetically modified to contain an exogenous nucleic acid encoding a bone morphogenetic factor and engrafted into bone, cartilage, or tooth tissue, e.g., to treat periodontitis. The cells optionally also include nucleic acids encoding other biologically active or therapeutic proteins or polypeptides, e.g., angiogenic factors, extracellular matrix proteins, cytokines or growth factors. For example, cells to be engrafted into pancreatic tissue contain a nucleic acid(s) encoding insulin or insulin precursor molecules. The cells also optionally include nucleic acids encoding gene products that decrease transplant rejection, e.g., CTLA4Ig CD40 ligand, or decrease development of transplant arteriosclerosis, e.g., inducible nitric oxide synthase (iNOS).

The invention also includes an apoptosis-resistant primary stem cell, e.g., an adult bone-marrow derived mesenchymal cell. The stem cell is genetically modified and includes an exogenous akt gene. Apoptosis of such a genetically-modified primary stem cell is reduced by at least 10% compared to a primary mesenchymal stem cell lacking the akt gene. Preferably, apoptosis is reduced by at least 50%, at least 2-fold, at least 5-fold, and up to at least 10-fold or more compared to a primary mesenchymal stem cell lacking the akt gene. Preferably, the stem cell is non-tumor forming. Although cells in which an exogenous akt gene sequence has been introduced produce increased amounts of an Akt gene product, the Akt protein is inactive under normoxia conditions. The Akt protein becomes activated upon exposure to hypoxia.

Also within the invention is method of increasing the viability and enhancing engraftment of transplanted stem cells. Stem cells to be transplanted are obtained from bone marrow tissue of an adult subject, genetically modified ex vivo, and then engrafted into the same or different recipient. Preferably, the donor and recipient are of the same species; more preferably, the donor and recipient are genetically similar (or the same) at major histocompatibility loci. For example, an autologous Transplant (self donor of bone marrow-derived mesenchymal stem cells), a syngeneic Transplant (identical twin donor). allogeneic transplant (related donor, unrelated donor, or “mismatched” donor) is performed. Transplanting Akt-modified cells leads to prolonged viability of the cells in the engrafted tissue. For example, the cells remain viable for 2, 3, 4, 5, 6, 7, 8, or more days and continue to grow and differentiate, whereas stem cells lacking akt sequences die in the peri-transplantation period, e.g., within 24 hours following transplantation.

The compositions and methods are useful for enhancing survival of grafted stem cells used in repairing or regenerating tissue, e.g., cardiomyocytes undergoing apoptosis due to an ischemic or reperfusion related injury; chondrocytes following traumatic injury to bone, ligament, tendon or cartilage; or hepatocytes in an alcohol-induced cirrhotic liver.

Disclosed are recombinant mesenchymal stem cells (rMSCs) that are genetically enhanced to have increased post-transplant survival when engrafted into striated cardiac muscle that has been damaged through disease or degeneration. Preferred rMSCs are recombinant for genes encoding a product that has an anti-apoptotic effect upon expression. Examples include the polypeptides encoded by the serine-threonine protein kinase Akt (i.e., protein kinase B, RAC-gamma protein kinase) gene (e.g., Akt-1, Akt-2, Akt-3), the heme oxygenase (HO) gene (e.g., HO-1, HO-2), the extracellular superoxide dismutase (ecSOD), and/or the interferon inducible dsRNA-activated protein kinase (PKR). A preferred gene is an isolated mammalian gene, and more preferably a human gene. Apoptosis may be inhibited directly through inhibition of functional apoptotic pathways or may be inhibited indirectly by increasing survivability of rMSCs under ischemic or hypoxic conditions.

The rMSCs differentiate into cardiac muscle cells and integrate with the healthy tissue of the recipient to replace the function of the dead or damaged cells, thereby regenerating the cardiac muscle as a whole.

In some embodiments, the rMSC is genetically engineered to express at least one, at least two, at least three, or more genes whose encoded polypeptides enhance survivability upon transplantation or engraftment.

Also disclosed is a composition containing a nucleic acid encoding a cytoprotective polypeptide, one or more oxygen sensitive regulatory elements that regulate the expression of the polypeptide, and a cell targeting expression element. Alternatively, the composition contains two, three, five, seven or ten oxygen sensitive regulatory elements. Preferably, the composition is administered to repair injury from an ischemic event such as a cardiac event, e.g., a myocardial infarction, stroke, hypertension, congestive heart failure, dilated cardiomyopathy, or restenosis.

The recipient subject may be suffering from or at risk of developing a condition characterized by aberrant cell damage such as oxidative-stress induced cell death (e.g., apoptotic cell death) or an ischemic or reperfusion related injury. A subject suffering from or at risk of developing a condition is identified by the detection of a known risk factor, e.g., gender, age, high blood pressure, obesity, diabetes, prior history of smoking, stress, genetic or familial predisposition, attributed to the particular disorder, or previous cardiac event such as myocardial infarction or stroke.

Conditions characterized by aberrant cell death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy.

The triggering agent or condition is endogenous or exogenous. All that is required is that the agent or condition induces the expression of the cell protective polypeptide. Preferably, induction is temporal. Induction of expression of the polypeptide occurs either pre-translation (e.g., via enhancers, promoters, response elements such as hypoxia or antioxidant response elements) or post-translation. For example, the condition is a physiological stimulus such as hypoxia, oxidative stress, reactive oxygen species such as hydrogen peroxide, superoxide or hydroxyl radicals. The agent is an antibiotic such as tetracycline; an immunosuppressive such as rapamycin; a steroid hormone such as ecdysone; or a hormone receptor antagonist such as mifepristone. Alternatively, the triggering agent is a member of a binary gene expression system such as the tetracycline responsive expression system or the ecdysone responsive expression system.

An oxygen sensitive regulatory element is an element that is modified by hypoxia or oxidative stress and is capable of regulating (e.g., turning on or turning off) expression of the cell protective polypeptide. For example, an oxygen sensitive regulatory element is a hypoxia-responsive element (HRE), an antioxidant response element (ARE) or an oxidative stress response element such as a peroxidase promoter or nuclear factor kappa B (NF-κB).

A cell targeting element is an element that is capable of restricting expression of the cell protective polypeptide to the cell type of interest, e.g., cardiac tissue or kidney tissue. For example a cell targeting element is a cell-specific promoter (e.g., α-MHC, myosin light chain-2, or troponin T).

To determine whether the composition inhibits oxidative-stress induced cell death, the composition is tested by incubating the composition with a primary or immortalized cell such as a cardiomyocyte. A state of oxidative stress of the cells is induced (e.g., by incubating them with hydrogen peroxide, i.e., H₂O₂) and cell viability is measured using standard methods. As a control, the cells are incubated in the absence of the composition and then a state of oxidative stress is induced. A decrease in cell death (or an increase in the number of viable cells) in the compound treated sample indicates that the composition inhibits oxidative-stress induced cell death. Alternatively, an increase in cell death (or an decrease in the number of viable cells) in the compound treated sample indicates that the composition does not inhibit oxidative-stress induced cell death. The test is repeated using different doses of the composition to determine the dose range in which the composition functions to inhibit oxidative-stress induced cell death.

In some embodiments, the nucleic acid compositions are formulated in a vector. Vectors include for example, an adeno-associated virus vector, a lentivirus vector and a retrovirus vector. Preferably the vector is an adeno-associated virus vector. Preferably the nucleic acid is operatively linked to a promoter such as a human cytomegalovirus immediate early promoter. An expression control element such as a bovine growth hormone polyadenylation signal is operably linked to coding region the cell protective polypeptide. In preferred embodiments, the nucleic acid of the invention is flanked by the adeno-associated viral inverted terminal repeats encoding the required replication and packaging signals. Nucleic acid compositions are inserted into a MSC through any suitable method known in the art.

The invention further features a method of treating a cardiac disorder in a subject with an rMSC composition expressing a nucleotide encoding a serine threonine kinase AKT polypeptide or a biologically active fragment thereof. A polypeptide fragment of a naturally occurring protein is at least 10 aa, at least 50 aa, at least 100 aa, at least 200 aa, at least 300 aa, at least 400 aa, at least 500 aa, at least 550 aa, up to and including a fragment that has one less amino acid than its respective full length polypeptide. A biologically active polypeptide of an AKT polypeptide has an amino acid sequence less than that of a naturally occurring AKT polypeptide, and that inhibits apoptosis-mediated cardiomyocyte death. The subject can be at risk if a cardiac disorder such as myocardial infarction, chronic coronary ischemia, arteriosclerosis, congestive heart failure, angina, atherosclerosis, and myocardial hypertrophy.

The invention further features a method of treating an acute or chronic cardiac disorder in a mammal suffering from or at risk of developing an acute or cardiac disorder by administering to the mammal a rMSC composition expressing a nucleotide encoding a human heme oxygenase polypeptide or a biologically active fragment thereof. A biologically active polypeptide of HO has an amino acid sequence less than that of a naturally occurring HO polypeptide and which inhibits oxidative stress-induced cardiomyocyte death. A chronic cardiac disorder includes disorders such as, chronic coronary ischemia, arteriosclerosis, congestive heart failure, angina, atherosclerosis, and myocardial hypertrophy.

The invention further features a method of treating a cardiac disorder in a subject with an rMSC composition expressing a nucleotide encoding an extracellular superoxide dismutase (ecSOD) polypeptide or a biologically active fragment thereof. A biologically active polypeptide of ecSOD polypeptide has an amino acid sequence less than that of a naturally occurring ecSOD polypeptide and which inhibits oxidative stress-induced cardiomyocyte death. The subject can be at risk if a cardiac disorder such as myocardial infarction, chronic coronary ischemia, arteriosclerosis, congestive heart failure, angina, atherosclerosis, and myocardial hypertrophy.

Also provided by the invention is a rMSC expressing a cardioprotective agent including a recombinant adeno-associated viral vector and a nucleotide encoding a human heme oxygenase-1 polypeptide or a human extracellular superoxide dismutase polypeptide or a human AKT polypeptide operatively linked to a human cytomegalovirus immediate early promoter. Preferably, the cardioprotective agent includes a bovine growth hormone polyadenylation signal. More preferably, the bovine growth hormone polyadenylation signal is flanked by the adeno-associated viral inverted terminal repeats.

Recombinant MSC cardiac muscle therapy is based, for example, on the following sequence: harvest of MSC-containing tissue, isolation and/or expansion of MSCs, transfection of MSCs with at least one anti-apoptotic gene, implantation of at least one rMSC into the damaged heart, and in situ formation of myocardium. This approach differs from traditional tissue engineering in that undifferentiated rMSCs are implanted and allowed to differentiate into their final form. Biological, bioelectrical and/or biomechanical triggers from the host environment may be sufficient, or under certain circumstances, may be augmented as part of the therapeutic regimen to establish a fully integrated and functional tissue.

Accordingly, one aspect of the present invention provides a method for producing cardiomyocytes in an individual in need thereof that comprises administering to said individual a sufficient amount of recombinant mesenchymal stem cells, allowing the cells to differentiate into myocardium, thus repairing damaged heart tissue.

The mesenchymal stem cells may be identified by specific cell surface markers. The surface markers of these isolated MSC populations are characterized as being 99% positive for connexin-43, c-kit (CD117) and CD90 and 100% negative for CD34, CD45, MHC, MLC, CTn1, αSA and MEF-2. A non-limiting method for the isolation of a population enriched in MSCs from primary bone marrow involves negative selection techniques against, e.g., cells positive for the CD34 cell surface marker, as described in the examples.

In some embodiments, an rMSC is induced in vivo to mobilize from the bone marrow to an ischemic heart by administering to a host subject a cytokine cocktail. In other embodiments, an rMSCs is implanted or transfused directly into a diseased heart or surrounding blood vessels. The administration of the cells can be directed to the heart by a variety of procedures. Localized administration is preferred. The mesenchymal stem cells can be from a spectrum of sources including, in order of preference: autologous, syngeneic, allogeneic or xenogeneic.

In one embodiment, the MSCs are administered as a cell suspension in a pharmaceutically acceptable medium for injection. Injection can be local, i.e. directly into the damaged portion of the myocardium, or systemic, i.e., injected into the peripheral circulatory system. Localized administration is again preferred.

In another embodiment, the rMSCs are further genetically modified or engineered to contain genes that express proteins of importance for the differentiation and/or maintenance of striated muscle cells. Also contemplated are genes that code for factors that stimulate angiogenesis and revascularization. Any of the known methods for introducing DNA are suitable, however electroporation, retroviral vectors and adeno-associated virus (AAV) vectors are currently preferred.

The invention also relates to the potential of MSCs to partially differentiate to the cardiomyocyte phenotype using in vitro methods. This technique can under certain circumstances optimize conversion of MSCs to the cardiac lineage by predisposing them the particular differentiation pathway. This has the potential for shortening the time required for complete differentiation once the cells have been administered.

Also within the invention is a method of enhancing migration, homing, adhesion, or engraftment of a cell to an injured tissue such as myocardial tissue. A cardiac injury or disorder includes myocardial infarction, congestive heart disease or failure. By homing is meant elaboration of a composition from the injured tissue, e.g., injured heart tissue, that recruits cells from the bone marrow or the circulation. By adhesion is meant binding of one cell to another or binding of a cell to an extracellular matrix. Adhesion encompases movement of cells, e.g., rolling, in blood vessels. Adhesion molecules are a diverse family of extracellular (e.g., laminin) and cell surface (e.g., NCAM) glycoproteins involved in cell-cell and cell-extracellular matrix adhesion, recognition, activation, and migration. Cell engraftment refers to the process by which cells, e.g., stem cells, become incorporated into a differentiated tissue and become part of that tissue. For example, stem cells bind to myocardial tissue, differentiate into functional myocardial cells, and become resident in the myocardium.

The method is carried out by increasing the amount of a polypeptide on the surface of the cell such as a stem cell. The method increases the number of stem cells in an area of injured tissue compared to the number of stem cells in the area in the absence of an exogenous stem cell-associated polypeptide or nucleic acid encoding such a polypeptide. The receptor is selected from the group consisting of CXCR4, IL-6RA, IL-6ST, CCR2, Selel, Itgal/b2, Itgam/b2, Itga4/b1, Itga8/b1, Itga6/b1, and Itga9/b1. Preferably, the cell is a stem cell such as a bone marrow-derived stem cell. More preferably, the cell is a mesenchymal stem cell. The amount of receptor on the surface of the cell is increase by contacting the cell with the protein or introducing into the cell a nucleic acid encoding said receptor under conditions that permit transcription and translation of the gene. The gene product is expressed on the surface of the stem cell. The stem cell receptor binds to a ligand that is expressed in injured tissue such as infarcted heart tissue.

A method of enhancing migration, homing, adhesion, or engraftment of a cell such as a stem cell to an injured tissue is carried out by increasing the amount of an injury-associated polypeptide, e.g., a cytokine or adhesion protein, in the injured tissue. The method increases the number of stem cells in an area of injured tissue compared to the number of stem cells in the area in the absence of an exogenous injury-associated polypeptide or nucleic acid encoding such a polypeptide. Identification of injury-associated polypeptides, e.g., growth factors, activate endogenous mechanisms of repair in the heart such as proliferation and differentiation of cardiac progenitor cells. For example, the injury-associated polypeptide is selected from the group consisting of SDF1, IL-6, CCL2, Sele, ICAM-1, VCAM-1, FN, LN, and Tnc. The injured tissue is cardiac tissue, such as ischemic myocardial tissue. The injured tissue is contacted with a nucleic acid encoding target protein or the protein itself, such as a cytokine or adhesion protein. For example, the target protein or a nucleic acid encoding the protein or is directly injected into the myocardium. Alternatively, cells such as fibroblast cells expressing exogenous nucleic acid molecules encoding the target proteins are introduced to the site of injury. The nucleic acid and amino acid sequences of the genes/gene products listed above are known and publically available, e.g., from GENBANK™.

The invention also relates to a method of diagnosing a cardiac disorder in a mammal suffering from or at risk of developing the cardiac disorder, by determining the levels of two of more genes that are differentially expressed during the cardiac disorder, or the polypeptides encoded thereby, in a patient derived sample, where an increase or decrease of these levels compared to normal control levels (i.e., a mammal not having the cardiac disorder) indicates that the mammal suffers from or is at risk of developing the cardiac disorder. The sample is derived from cardiac tissue, blood, plasma or serum.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a schematic representation of the method for isolation of bone marrow derived mesenchymal stem cells.

FIG. 2 is a graphic representation of proliferation characteristics of bone marrow stromal cells.

FIG. 3 depicts a immunohistochemical analysis of surface markers of isolated mesenchymal stem cells.

FIG. 4 is a gel analysis of RT-PCR results confirming surface marker expression in mesenchymal stem cells.

FIGS. 5A-5D represent schematics of high efficiency retroviral gene transfer vector for use in mesenchymal stem cells, and transfection efficiencies for each vector.

FIG. 6 depicts 5 μm thick cardiac tissue sections injected with mesenchymal stem cells or control.

FIG. 7 depicts X-gal staining of 2 mm thick cardiac tissue sections injected with nLacZ transfected mesenchymal stem cells or control vehicle.

FIG. 8 is a 10× magnification of 5 μm thick cardiac tissue sections stained with X-gal to observe nLacZ transfected mesenchymal stem cells or control vehicle.

FIG. 9 is a 40× magnification of 5 μm thick cardiac tissue sections stained for green fluorescent protein (GFP) to observe GFP transfected mesenchymal stem cells or control vehicle.

FIGS. 10A-10F demonstrated co-localization of staining for engrafted GFP transfected mesenchymal stem cells and cardiomyocyte specific cell markers at three weeks post-transfection.

FIGS. 11A-11D depict TUNEL and cell characterization results that determine the degree of protection against apoptosis provided to engrafted recombinant mesenchymal stem cells ectopically expressing Akt.

FIG. 12A depicts TUNEL results for rMSCs co-expressing GFP and Akt. FIG. 12B is a histographic depiction of the TUNEL results.

FIGS. 13A and 13B are histographic representations of areas at risk in treated hearts and volume of remaining infarcted myocardium after injection with various amounts of recombinant mesenchymal stem cells expressing, e.g., Akt, LacZ, c-kit, or saline control.

FIG. 14 depicts cross sections of infarcted hearts injected as shown in FIGS. 13A and 13B, compared to sham treated controls.

FIG. 15 is a histographic depiction of the volume of regenerated myocardium in infarcted hearts treated as described in FIGS. 13A and 13B.

FIGS. 16A and 16B are histographic representations of left ventricular end systolic pressure baselines, and of rate of relaxation, respectively, in hearts treated as described in FIGS. 13A and 13B.

FIGS. 17A-H is a series of photographic images demonstrating the immunocytochemical characterization of MSCs of the present invention.

FIGS. 18A and 18B are bar graphs depicting differentially expressed genes following myocardial infarction. Gene expression was determined by RT-PCR in infarcted tissue (MI) compared to sham at 24 hours.

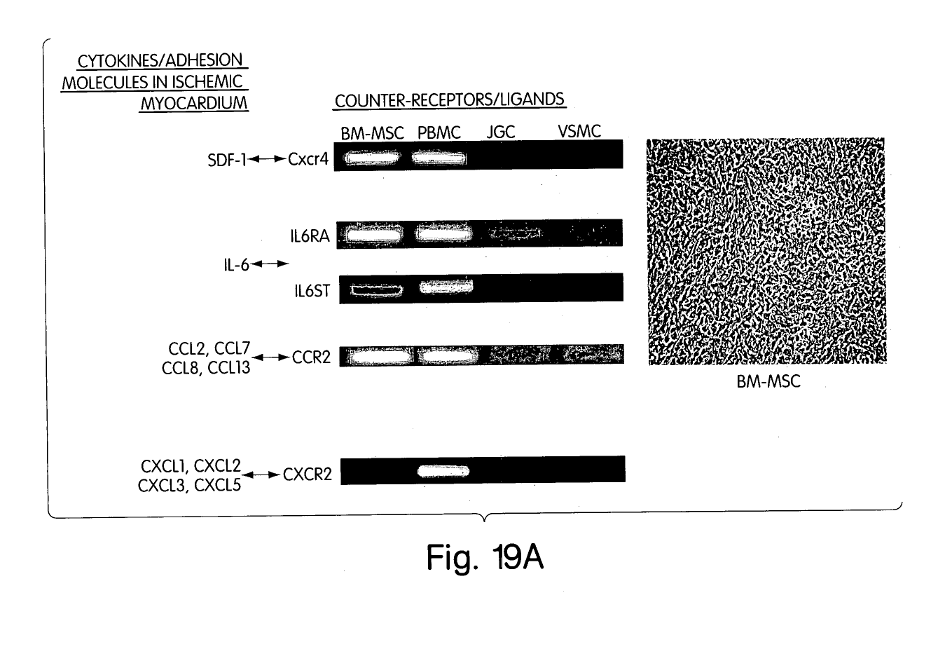

FIGS. 19A-B is a photograph showing the results of a RT-PCR analysis of receptors/ligands in BMSC (P1, passage 1; P6, passage 6), peripheral blood mononuclear cells (PBMC), juxtaglomerular cell (JGC) and vascular smooth muscle cells (VSMC). Abbreviations in FIG. 19A include SDF1, stromal derived factor 1; CXCR4, chemokine (C-X-C motif) receptor; IL6, interleukin-6; IL6RA, interleukin-6 receptor alpha; IL6ST, IL6 signal transducer, CC, chemokine (C-C motif); CXC, chemokine (C-X-C motif); CCR, CC receptor. Abbreviations in FIG. 19B include SDF1, stromal derived factor 1 FIG. 19B is a photograph of

### DETAILED DESCRIPTION

Mesenchymal stem cells (MSCs) are progenitor cells known to have a broad potential for cellular differentiation into more than one type of cell lineage and have a greatly reduced incidence of immune system-mediated rejection when grafted into non-autologous hosts. MSCs have a demonstrated ability to differentiate into cardiomyocytes, vascular endothelia and connective tissue. See, e.g., Pittenger et al., 1999 Science 284: 143-147; U.S. Pat. Nos. 6,387,369, 6,214,369, 5,906,934, 5,827,735, 5,591,625, 5,486,359, and 5,197,985.

The bone marrow of an adult animal is a repository of mesenchymal stem cells (MSCs). These cells are self-renewing, clonal precursors of non-hematopoietic tissues. They are multi-potent. MSCs can differentiate into osteoblasts, chondrocytes, glial cells, astrocytes, neurons and skeletal muscle. Cells isolated from bone marrow can differentiate into blood vessels and capillaries. For example, bone marrow-derived mononuclear cells (BM-MNCs), when transplanted into myocardial ischemic tissue and skeletal muscle ischemic tissue, form new blood vessels and increase angiogenesis in said target tissue. See, PCT publication WO 02/08389. Bone marrow derived stem cells can differentiate into cardiac muscle, and are useful for restoration of cardiac function.

Oxidative stress has been shown to be the major cause of death for cells grafted into injured myocardium. Wang, et al. 2001, J Thorac Cardiovasc Surg 122: 699-705; Zhang et al., 2001, J Mol Cell Cardiol 33: 907-921. Transgenic cells that are recombinant for cytoprotective genes such as the serine-threonine protein kinase Akt (protein kinase B) and heme oxygenase (HO) protect cells against ischemic injury and increase graft cell survival when grafted into infarcted myocardial scar tissue.

A cell protective (i.e., cytoprotective) polypeptide is a polypeptide that is capable of inhibiting cell damage such as oxidative-stress induced cell death. Suitable tissue protective polypeptides include, as non-limiting examples, an antioxidant enzyme protein, a heat shock protein, an anti-inflammatory protein, a survival protein, an anti-apoptotic protein, a coronary vessel tone protein, a pro-angiogenic protein, a contractility protein, a plaque stabilization protein, a thromboprotection protein, a blood pressure protein and a vascular cell proliferation protein. Preferably the cell protective polypeptide is a human Akt polypeptide (e.g., Akt-1, Akt-2 or Akt-3), a human heme oxygenase polypeptide (e.g., HO-1 or HO-2), a human interferon-inducible double-stranded RNA-activated protein kinase (i.e., PKR; eukaryotic translation initiation factor 2 alpha protein kinase 2; P1/eIF-2A protein kinase) polypeptide or a human extracellular superoxide dismutase (i.e., ecSOD), or a biologically active fragment of any such polypeptide. Exemplary human Akt-1 polypeptides includes for example GenBank Accession numbers NP_{—}005154 and AAH00479. Exemplary human Akt-2 polypeptides includes for example GenBank Accession numbers P31751 and NP_{—}001617. Exemplary human Akt-3 polypeptides includes for example GenBank Accession numbers Q9Y243 and NP_{—}005456. Exemplary human heme oxygenase-1 polypeptides includes for example GenBank Accession numbers P09601 and CAA32886. Exemplary human heme oxygenase-2 polypeptides includes for example GenBank Accession numbers P030519 and AAH02396. Exemplary human extracellular superoxide dismutase polypeptides includes for example GenBank Accession numbers Q07449 and P08294. Exemplary human PKR polypeptides includes for example GenBank Accession numbers P19525, JC5225 and NP_{—}002750.

Other cytoprotective genes are provided in Table 1.

Mesenchymal Bone-Marrow Cells

Bone marrow-derived mesenchymal stem cells differentiate into a variety of cell types including cardiac myocytes, osteoblasts, chondrocytes, astrocytes, pneumocytes and neurons. Systemically administered MSCs home and migrate towards specific organs, e.g., the brain, where they engraft in and migrate within the brain to form astrocyte-type grafts, acquiring a neuronal phenotype with expression of neuron specific markers NeuN and MAP-2 and GFAP and improve functional outcome. In addition, bone marrow-derived cells differentiate into skeletal muscle satellite cells, and mature skeletal muscle, e.g., in an animal model of Duchenne's muscular dystrophy, and into type I pneumocytes in recipients that had sustained bleomycin induced lung injury. MSCs also differentiate into myocardial cells in regions of myocardial infarct.

Ex vivo genetic manipulation is carried out using known methods prior to transplantation. MSCs are autologous or syngeneic. Alternatively, the MSCs are allogeneic. Allogeneic rMSCs are optionally modified to prevent or decrease any immune response from the donor.

Peri-Transplantation Mesenchymal Stem Cell Survival is Enhanced by Genetic Modification with Akt

Prior to the invention, regenerative capacity is limited by cell death in the peri-transplantation period. Although the primary cause behind peri-transplant cell death is thought to be placement of cells into an ischemic environment devoid of nutrients and oxygen, inflammation, the loss of survival signals from matrix attachments or cell-cell interactions, and the actual mechanics of transplantation all contribute to increased apoptosis. The methods described herein enhance the viability of transplanted cells through genetic engineering. Akt is activated by hypoxia, oxidative stress, fluid shear, inflammatory cytokines such as TNF-alpha, and a variety of other growth factors and cytokines. Akt is a general mediator of survival signals, and is both necessary and sufficient for cell survival. It achieves this by targeting apoptotic family members Ced-9/Bcl-2 and Ced-3/caspases, forkhead transcription factors, IKK-alpha and IKK-beta, and plays a role in modulating intracellular glucose metabolism, e.g., by increasing glucose transportation. Akt promotes MSC viability both in vitro and in the early post-transplant period. Use of wild-type Akt, which was not constitutively expressed, but was activated when needed, protected cells from apoptosis, while avoiding the potential detrimental effects of constitutive activated −Akt expression. As a result, intra-cardiac retention, engraftment and differentiation of MSCs genetically enhanced to over-express Akt was superior to that of control MSCs (e.g., those expressing reporter genes alone).

In the cardiac transplantation model, retention of greater numbers of MSCs due to increased longevity/viability in the ischemic myocardium led to a greater volume of regenerated myocardium after 3 weeks, normalization of systolic and diastolic cardiac function, and prevention of remodeling.

Nucleic acids encoding an Akt gene product were introduced to cells by retroviral transduction. Transduction efficiencies of over 80% were observed after MSCs in culture were exposed to high titer retroviral supernatant between days 10 and 15, and prior to separation from the hematopoietic fraction using retroviruses expressing either GEP or Lac Z. The cells continued to proliferate in culture and continued to express stem cell marker c-kit after genetic manipulation. A Murine Stem Cell Virus (pMSCV) from Clontech was used, thereby circumventing a potential issue with retroviral silencing after transplantation. The retroviral vector achieved stable, high-level gene expression. Gene expression was observed for the duration of our experiment (8 weeks in vitro, and 3 weeks in vivo).

Retrovirally transduced MSCs were transduced with the prosurvival serine-threonine kinase Akt. At baseline conditions, of 37° C. and 21% ambient O₂, Akt activity was equivalent in both groups. After 24 hours of hypoxia in serum-free medium, Akt activity increased 28.5-fold in the Akt-MSC group, and 6.6-fold in hypozin in serum-free medium, Akt activity increased 28.5-fold in the Akt-MSC group, and 6.6-fold in he GFP-MSC group, reducing MSC apoptosis by 79%, and reducing DNA laddering. The protective effects of Akt were assessed in vivo by double-staining left ventricular sections for c-kit⁺ and TUNEL or annexin-V. This method allowed a determination of the number of c-kit⁺ cells retained in the myocardium, and the percent of c-kit⁺ cells that were apoptotic. Twenty-four hours after transplantation, of 5×10⁶ LacZ-MSCs into ischemic myocardium, 68% of 33±1.53 LacZ-MSCs per high power field (hpf) were apoptotic. By contrast, twenty-four hours after transplantation of 5×10⁶ Akt-MSCs only 19% of 82+6.7 Akt-MSCs per hpf were apoptotic (p<0.001). An additional forty-eight hours later, 31% of 22.7+9.8 LacZ-MSCs per hpf were apoptotic; whereas 17% of 66+3.5 Akt-MSCs per hpf were apoptotic (p<0.001). There were no c-kit⁺ cells present in the myocardium after three weeks. These observations indicate that Akt is activated in MSCs exposed to hypoxia and serum-starvation in vitro, as well as after transplantation into the ischemic myocardium, and that increased Akt activity prevents MSCs apoptosis in the immediate post-implantation period, e.g., 1, 2, 3, 4, 5, 7, days and several weeks post-transplantation.

Expression of Exogenous Polypeptides

MSCs are genetically modified to express exogenous nucleic acids encoding one or more cell surface receptors. These receptors include CxC chemokine receptors (e.g., CxCr1-6), CC chemokine receptors (e.g., CC12, CC16, CC17 and CC19); interleukin receptors; trk receptors; estrogen receptors; integrin receptors; tumor necrosis factor (TNF) receptor; other chemokine receptors (e.g., fekL; Fek-1); vascular endothelial cell growth factor receptor (VEGF-R, e.g., Flt-1, Flk1); ephrin receptors (EPHs), IgG receptors (e.g., IgGa4 and IgGb1); and platelet-derived growth factor receptors.

The present invention also provides rMSCs that express one or more adhesion molecules. These adhesion molecules include P-selectin, E-selectin, vascular cell adhesion molecule (VCAM), intracellular adhesion molecule (ICAM), platelet-endothelial cell adhesion molecule (PECAM), and LF-1.

The present invention also provides rMSCs that express one or more extracellular matrix (ECM) proteins on the cell surface, optionally in combination with one or more modulators of extracellular matrix proteins. Exemplary extracellular matrix proteins include integrins, fibronectin, collagens, laminin, tenascin C, vitronectin CSPG, and thrombospondin. Exemplary ECM modulatory proteins include matrix metalloproteases (MMPs), MT-MMPs, tissue inhibitors of metalloproteases (TIMPs), dispase, collagenase, and EMMPRIN.

The present invention also provides rMSCs that express one or more growth factors or cytokines, including SDF-1, interferons, interleukins, heparin, tissue plasminogen activator, TNF, transforming growth factor (TGF), platelet factor (e.g., PF-4), insulin-like growth factors (IGFs), hepatocyte growth factor (HGF), epithelial cell growth factor (EGF), erythropoietin, Ephrins, and colony-stimulating factors (CSFs).

In addition to the proteins described above, MSCs include exogenous nucleic acids that express one or more antioxidant proteins. Exemplary anti-oxidants include superoxide dismutase, heme oxygenase-1 (HO-1), ATX-1, ATOX-1, and AhpD.

Genes encoding one or more inducers of angiogenesis and/or vasculogenesis are optionally transduced into the cells as well. Such inducers include VEGF, fibroblast growth factors (FGF), PDGF, Ephrins and hypoxia-inducible factors (HIFs).

rMSC Differentiation

rMSCs differentiate into specific cell types within a selected target tissue. In the myocardium, rMSCs differentiate into, e.g., cardiomyocytes. In the brain or spinal column, rMSCs differentiate into neurons and/or astrocytes. In bone, rMSCs differentiate into osteoblasts, osteoclasts, or osteocytes. In cartilage, rMSCs differentiate into chondrocytes. In adipose tissue, rMSCs differentiate into adipocytes. In skeletal muscle, rMSCs differentiate into myocytes or satellite cells. In the liver, rMSCs differentiate into hepatocytes. In the lung, rMSCs differentiate into pneumocytes. In blood vessels, rMSCs differentiate into endothelial cells, smooth muscle cells, or pericytes.

Tissue-Specific Delivery Systems

MSCs are capable of differentiating into a number of cell types. The present invention encompasses delivery systems in which rMSCs are administered systemically or locally to colonize a selected type of tissue, e.g., an injured tissue. For example, rMSCs are directly injected into the target tissue. The injection site is at a site of injury, or nearby the injured tissue. Alternatively, rMSCs expressing a specific recombinant ligand or receptor are introduced to the subject and then the cells targeted to a desired target tissue by inducing expression of the cognate binding partner in the target tissue.

MSC are modified to only produce anti-apoptotic protein (Akt) in specific tissue. By way of non-limiting example, the exogenous nucleic acid that includes the Akt gene is placed under the control of a tissue-specific promoter. Alternatively, Akt expression is placed under the control of a light-sensitive promoter; whereby the Akt gene is expressed only in tissues or regions thereof illuminated in a controlled manner.

Prevention of Ischemia-Reperfusion Injury by Pre-Injury Contact with rMSCs

Situations arise in a clinical setting wherein cell death and tissue damage caused by ischemia-reperfusion can be reasonably predicted to occur as a result of certain surgical procedures. Cardiac procedures that may result in ischemia-reperfusion injury include balloon angioplasty, coronary bypass surgery, heart transplantation, and valve replacement surgery. Similar damage occurs in the kidney, liver, and other organs resulting from decrease or cessation of blood flow. Systemic or multi-organ ischemia-reperfusion damage may also result from hypothermia, infection, and other causes. The present invention encompasses methods of preventing or reducing ischemia-reperfusion cell death and tissue damage by treating the subject with rMSCs prior to and/or concomitant with the injury.

rMSCs Containing Two or More Exogenous Gene Sequences

The present invention provides for rMSCs that contain two or more exogenous gene sequences. These gene sequences may be operably linked to a single promoter, or two promoters, and may be contained in the same nucleic acid (in cis) or on separate nucleic acids (in trans). Gene sequences as used herein include nucleic acids encoding an open-reading from of a protein, or a portion of an open-reading frame such that the translated polypeptide has biological activity similar to that of the polypeptide translated from the complete open-reading frame. Gene sequences also include promoters, enhancers, and silencing elements.

The two or more gene sequences are an anti-apoptotic gene (e.g., Akt) and a cell surface receptor (e.g., a homing molecule); an anti-apoptotic gene and an adhesion molecule; an anti-apoptotic gene and a growth factor; an anti-apoptotic gene and an anti-oxidant; an anti-apoptotic gene and an angiogenesis/vasculogenesis inducer; or an anti-apoptotic gene and an extracellular matrix protein or an ECM modulator.

Cytokines and Adhesion Receptors Mediate Trafficking, Homing and Engraftment of MSCs into Injured Tissue

Specific cytokines and adhesion receptors play a critical role in homing and adherence of MSCs to damaged tissue, such as myocardium injured by ischemia-reperfusion. The present invention provides for the enrichment of MSCs or the generation and use of rMSCs that express exogenous levels of these cytokines and adhesion receptors. By way of non-limiting example, MSCs that express a specific collection of cell surface receptors and ligands are enriched using cell sorting, and are genetically modified both these and, optionally, non-enriched MSCs, using high-efficiency retroviral gene transfer strategies. These rMSCs have increased responsiveness to the cytokines generated from the ischemic heart and increased adhesion to ischemic myocardium, which in turn increases engraftment. For example, introduction of the IL-8 receptor into rMSCs is useful for homing, and α-integrin 4 is useful for adhesion.

Cell-Marker Characterization of MSCs

Isolated MSCs are distinguished from other cell types on the basis of presence of markers, such as cell surface polypeptides. Detection of these markers can be performed using immunocytochemistry, FACS sorting, and RT-PCR. Useful markers of the MSC type include:

a. Growth Factor Receptors: CD121 (IL-1R), CD25 (IL-2R), CD123 (IL-3R), CD71 (Transferrin receptor), CDI17 (SCF-R), CD114 ((3-CSF-R), PDGF-R and EGF-R

b. Hematopoietic markers: CD1a, CD11b, CD14, CD34, CD45, CD133

c. Adhesion receptors: CD166 (ALCAM), CD54 (ICAM-1), CD102 (ICAM-2), CD50 (ICAM-3), CD62L (L-selectin), CD62e (E-selectin), CD3I (PECAM), CD44 (hyaluronate receptor)

d. Integrins: CD49a (VLA-α1), CD49b(VLA α2), CD49c (VLA-α3), CD49d (VLA-α4), CD49e (VLA α5), CD29 (VLA-β), CD 104 (β4-integrin).

e. Other miscellaneous markers. D90 (Thyl), CD105 (Endoglin), SH-3, SH-4, CD80 (B7-1) and CD8 (B7-2)

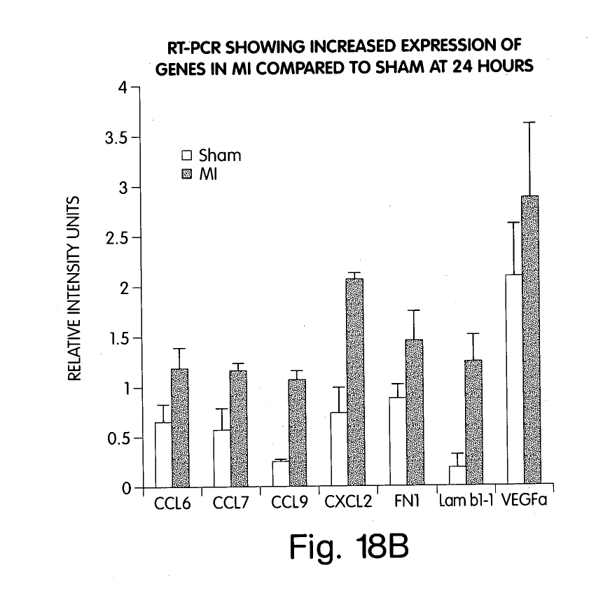

Specific collections (or “signatures”) of MSC markers are provided, which allow the generation of rMSCs that are capable of differentiating into specific cell types. By way of non-limiting example, a sub-population of MSCs with the greatest capacity to develop into cardiac myocytes can be isolated using a cardiac myocyte signature. An example of immunocytochemical characterization of MSCs of the present invention is provided in FIGS. 17A-H.

Coronary Disorders

Many patients are either at risk for or have suffered from various types of heart failure, including myocardial infarction, symptomatic or unsymptomatic left ventricular dysfunction, or congestive heart failure (CHF). An estimated 4.9 million Americans are now diagnosed with CHF, with 400,000 new cases added annually. This year over 300,000 Americans will die from congestive heart failure. Cardiac muscle does not normally have reparative potential. The ability to augment weakened cardiac muscle would be a major advance in the treatment of cardiomyopathy and heart failure. Despite advances in the medical therapy of heart failure, the mortality due to this disorder remains high, where most patients die within one to five years after diagnosis.

Coronary disorders, can be categorized into at least two groups. Acute coronary disorders include myocardial infarction, and chronic coronary disorders include chronic coronary ischemia, arteriosclerosis, congestive heart failure, angina, atherosclerosis, and myocardial hypertrophy. Other coronary disorders include stroke, myocardial infarction, dilated cardiomyopathy, restenosis, coronary artery disease, heart failure, arrhythmia, angina, or hypertension.

Acute coronary disorders result in a sudden blockage of the blood supply to the heart which deprives the heart tissue of oxygen and nutrients, resulting in damage and death of the cardiac tissue. In contrast, chronic coronary disorders are characterized by a gradual decrease of oxygen and blood supply to the heart tissue overtime causing progressive damage and the eventual death of cardiac tissue.

Tissue Protective Polypeptides

Table I provides a list of tissue protective polypeptides useful in the compositions and methods of the invention.

TABLE I
Targets for gene-based therapy for congenital and acquired heart disease.

| Strategy | Therapeutic Target | Genetic manipulation | Vector | Application |
|---|---|---|---|---|
| Protection/Prevention | | | | |
| Antioxidant enzymes | HO-1, SOD, catalase, GPx | overexpression | AAV, LV | CAD, MI |
| Heat shock proteins | HSP70, HSP90, HSP27 | overexpression | AAV, LV | CAD, MI |
| Anti-inflammatory | I-CAM, V-CAM, | inhibition | AS-ODN | graft |
| | NF-κB, TNF-α | | Decoy ODN | atherosclerosis, |
| | | | AAV-AS-ODN | transplantation |
| | | | RV-AS-ODN | |
| Survival genes | Bcl-2, Akt | overexpression | AAV, LV | CAD, MI, HF |
| Pro-apoptotic genes | Bad, p53, Fas ligand | inhibition | AS-ODN | MI, HF |
| | | | Decoy ODN | |
| | | | AAV-AS-ODN | |
| Coronary vessel tone | eNOS, adenosine | overexpression | RV, AAV | CAD, HF |
| | (P1, P3) receptors | | | |
| Rescue | | | | |
| Pro-angiogenic genes | VEGF, FGF, HGF | overexpression | AAV | CAD, MI, HF |
| Contractility | β-adrenergic receptors, | overexpression | AAV | HF |
| | SERCA 2A, V1 receptor | | | |
| | BARK, Phosphalamban | Inhibition | AAV | HF |
| Plaque stabilization | CD40 | overexpression | RV, AAV(?) | CAD |
| Thromboprotection | PAI-1, plasminogen | inhibition | AS-ODN | CAD, MI |
| | activator Tissue factor | | | |
| | TPA, hirudin, urokinase | overexpression | AAV | CAD, MI |
| | Thrombomodulin, | | | |
| | COX-1, PGI₂ synthase | | | |
| Blood pressure | Kallikrein, eNOS, ANP | overexpression | AAV, RV | hypertension, |
| | ACE, AGT, AT₁ | inhibition | AAV-AS-ODN | HF |
| Vascular cell | NOS, Ras dominant | overexpression | AD, RV, AAV | graft |
| proliferation | negative E2F, c-myb, | inhibition | AS-ODN, | atherosclerosis, |
| | c-myc, PCNA | | Decoy-ODN | restenosis |
| Inherited heart disease | | | | |
| Channelopathies | SCN5A, Iₖ | overexpression | α-MHC-AAV | arrhythmia |
| Cardiomyopathy | sarcomeric proteins, | overexpression | α-MHC-AAV | DCM |
| | sarcoglycans | | | (in utero) |
| Abbreviations: | | | | |
| AAV, adeno-associated virus; AS-ODN, antisense oligodeoxynucleotide; CAD, coronary artery disease; DCM, dilated cardiomyopathy; HF, heart failure; LV, lentivirus; MI, myocardial infarctionα-MHC, alpha myosin heavy chain; RV, retrovirus, HO-1, Heme oxygenase-1; SOD, superoxide dismutase; GPx, glutathione peroxidase; HSP70, 70 kD heat shock protein; HSP90, 90 kD heat shock protein; HSP27, 27 kD | | | | |
| # heat shock protein; I-CAM, intercellular adhesion molecule; V-CAM, vascular adhesion molecule; NF-κB, nuclear factor kappa B; TNF-α, tumor necrosis factor alpha; eNOS, endothelial nitric oxide synthase; VEGF, vascular endothelial growth factor; FGF, fibroblast growth factor; HGF, hematopoietic growth factor; SERCA 2A, sarcoplasmic/endoplasmic reticulum Ca-2+ ATPase; V1 receptor, vasopressin-1 | | | | |
| # receptor; bARK, beta-adrenergic receptor kinase; PAI-1, plasminogen activator inhibitor-1; TPA, tissue plasminogen activator; COX-1, cyclooxygenase-1; PGI₂ synthase, prostacyclin synthase; ANP, atrial natriuretic peptide; ACE, angiotensin-converting enzyme; AGT, angiotensinogen; AT₁, Angiotensin II-type-1; NOS, nitric oxide synthase; PCNA, proliferating cell nuclear antigen; SCN5A, cardiac sodium channel gene 5A. | | | | |

Regulatable Gene Expression

Effective gene therapy requires that gene expression is regulated in order to achieve optimal expression levels and reduce side effects associated with constitutive gene expression. An ideal strategy for myocardial protection against ischemia/reperfusion injury with minimal potential side effects resulting from constitutive expression of the transgene is a regulatable expression system. In some embodiments, turn on gene expression would occur with the onset of ischemia (hypoxia), so that the gene product is already present during reperfusion.

Many transcription factors are modified by hypoxic and oxidative stress. Studies of molecular responses to hypoxia have identified HIF-I as the master regulator of hypoxia-inducible gene expression. Under hypoxic conditions, HIF-I binds to the hypoxia-responsive element (HRE) in the enhancer region of its target genes and turns on gene transcription. Additionally, reperfusion or reoxygenation after ischemia increases the transactivating ability of NF_{K}B Genes regulated by NF_{K}B include cytokines and adhesion molecules, which contribute to cell death by promoting inflammatory responses. Several studies indicate that the hypoxic and hyperoxic environment can be used to activate heterologous gene expression driven by HRE and cis-acting consensus sequences of activated NF_{K}B respectively. Accordingly, in one aspect of the invention, at least one HRE is utilized as an enhancer to drive transgene expression. To assure sufficient duration of the transgene expression to achieve myocardial protection during the reperfusion period, as second regulatory element that is activated by oxidative stress such as NF_{K}B responsive element is utilized in certain embodiments.

Cell Specific Gene Expression

The potential applications of gene therapy are currently limited by the absence of efficient cell-specific targeting vectors. This lack of tissue specificity is a fundamental problem for gene therapy as proteins that are therapeutic in target cells also may be harmful to normal tissue. Thus non cell-specific expression of a transgene has the potential for inducing metabolic and physiologic mechanisms that could result in pathology over the long term. Localized injections can provide certain degree of localized expression of the targeting vector, however, there may still be a spill over into the circulation which will affect other cells and organs. One way to circumvent this problem is to use transcriptionally targeted vectors that can restrict the expression of the therapeutic proteins primarily to the target cells by the use of tissue-specific promoters (e.g. a-myosin heavy chain, myosin light chain. The cells in the myocardium that are particularly prone to reperfusion injury are the cardiac myocytes and microvascular endothelial cells. Thus, a cell specific strategy could be directed to protect either cell type.

Myocardial Protection with HO-1, Akt or ecSOD Gene Expression

The selection of HO-1 as a therapeutic agent was made on the basis of evidence that the enzyme neutralizes the potent pro-oxidant activity of heme and that its multiple catalytic by-products bilirubin, carbon monoxide (CO) and free iron together exert powerful, pleiotropic cytoprotective effects. Bilirubin is a potent endogenous antioxidant that scavenges peroxyl radicals and reduces peroxidation of membrane lipids and proteins. CO is a vasodilator and powerful anti-inflammatory and antiapoptotic agent. Free iron stimulates the synthesis of the iron binding protein ferritin, which reduces iron-mediated formation of free radicals and upregulates several key cytoprotective genes.

Recombinant Cell Therapy to Cardiac Tissue

Gene therapy refers to therapy that is performed by the administration of a specific nucleic acid to a subject. A nucleic acid is delivered to a target cell that in turn produces a gene product that exerts a therapeutic effect, e.g., inhibition of cell damage such as cardiomyocyte death after a hypoxia-related injury. Standard gene therapy methods known in the art may be used in the practice of the present invention. See, e.g., Goldspiel, et al., 1993. Clin Pharm 12: 488-505. Recombinant cell therapy refers to therapy that is performed by the administration of a genetically modified autologous or heterologous cell to a subject.

A therapeutic composition of the invention contains at least one MSC expressing a recombinant nucleic acid encoding an anti-apoptosis polypeptide operably linked to a promoter. Insertion of the nucleic acid into a MSC may be with any suitable vector known to one skilled in the art. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. Suitable expression vectors, include viral vectors (e.g., replication defective retroviruses, adenoviruses and adeno-associated viruses). Additionally, some viral vectors are capable of targeting a particular cells type either specifically or non-specifically.

The recombinant expression vectors contain a nucleic acid in a form suitable for expression in a target cell, e.g., myocardium cell. Recombinant expression vectors include one or more regulatory sequences, operatively linked to the nucleic acid sequence to be expressed. For example, the vector includes a promoter and/or an enhancer sequence which preferentially directs expression of a nucleic acid in vascular, e.g., cardiac-restricted ankyrin repeat protein promoter. Operably linked is means that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are known in the art. See, Goeddel; GENE EXPRESSION TECHNOLOGY: METHODS IN ENZYMOLOGY 185, Academic Press, San Diego, Calif. (1990).

The promoter may be inducible or constitutive, and, optionally, tissue-specific. The promoter may be, e.g., viral or mammalian in origin. Preferably the promoter is a human cytomegalovirus immediate early promoter. A nucleic acid molecule composition contains an expression control element that is operably-linked to coding region(s) of a cell protective polypeptide (e.g., hHO-1 polypeptide or an ecSOD polypeptide). In some embodiments, the expression control element is a bovine growth hormone polyadenylation signal. In certain embodiments, a polypeptide encoding a nucleic acid molecule and regulatory sequences are flanked by regions that promote homologous recombination at a desired site within the genome, thus providing for intra-chromosomal expression of nucleic acids. For example, the nucleic acid molecule is flanked by the adeno-associated viral inverted terminal repeats encoding the required replication and packaging signals. See e.g., Koller and Smithies, 1989. Proc Natl Acad Sci USA 86: 8932-8935. Alternatively, a nucleic acid remains episomal and induces an endogenous gene, e.g., an endogenous HO gene.

Delivery of the rMSC into the heart of a patient may be either direct (i.e., injection in vivo of a rMSC to patient cardiomyocyte tissues) or indirect (i.e., perfusion of rMSCs into the peripheral blood vessel of a subject, with subsequent homing of the rMSC to the injured cardiac tissue). The nucleic acid may be delivered to a MSC cell by a viral vector (e.g., by infection using a defective or attenuated retroviral or other viral vector; see U.S. Pat. No. 4,980,286); by directly injecting naked DNA; by using microparticle bombardment (e.g., a “Gene Gun®; Biolistic, DuPont); by coating the nucleic acids with lipids; by co-administering a cell-surface receptors/transfecting agents; by encapsulating the nucleic acid in liposomes, microparticles, or microcapsules; or by linking the composition to a peptide that is known to enter the nucleus. In certain embodiments, nucleic acid compositions are associated with a ligand that facilitates receptor-mediated endocytosis (see, e.g., Wu and Wu, 1987. J Biol Chem 262: 4429-4432), to “target” cell types that specifically express the receptors of the linked ligand.

Gene Therapy Vectors for rMSCs

Prior to the in vivo administration of the resulting recombinant cell, the nucleic acid is introduced into a cell by any method known within the art including, but not limited to transfection, electroporation, microinjection, infection with a viral or bacteriophage vector containing the nucleic acid sequences of interest, cell fusion, lipofection, calcium phosphate-mediated transfection, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. In some embodiments, the methodology of transfer includes the concomitant transfer of a selectable marker to the cells. The cells are then placed under selection pressure (e.g., antibiotic resistance) so as to facilitate the isolation of those cells that have taken up, and are expressing, the transferred gene. The gene transfer method leads to stable transfer of the nucleic acid to the cell; i.e., the transferred nucleic acid is heritable and expressible by the cell progeny. Those cells are then delivered to a patient.

The resulting recombinant cells are delivered to a patient by various methods known within the art including, but not limited to, injection of transfected cells (e.g., subcutaneously) or directly into cardiac tissue. For example, HO nucleic acid constructs are introduced into autologous or histocompatible epithelial cells and recombinant skin cells are applied as a skin graft onto the patient. In some embodiments, 5×10⁶ rMSCs are injected into the treatment site. Numbers of rMSCs injected per treatment site may be at least 1×10⁴ cells, at least 2.5×10⁴ cells, at least 5×10⁴ cells, at least 7.5×10⁴ cells, at least 1×10⁵ cells, at least 2.5×10⁵ cells, at least 5×10⁵ cells, at least 7.5×10⁵ cells, at least 1×10⁶ cells, at least 2.5×10⁶ cells, at least 5×10⁶ cells, at least 7.5×10⁶ cells, at least 1×10⁷ cells, at least 2.5×10⁷ cells, at least 5×10⁷ cells, at least 7.5×10⁷ cells, or at least 1×10⁸ cells.

The concentration of cells per unit volume, whether the carrier medium is liquid or solid remains within substantially the same range. The amount of MSCs delivered will usually be greater when a solid, “patch” type application is made during an open procedure, but follow-up therapy by injection will be as described above. The frequency and duration of therapy will, however, vary depending on the degree (percentage) of tissue involvement (e.g. 5-40% left ventricular mass).

In cases having in the 5-10% range of tissue involvement, it is possible to treat with as little as a single administration of rMSC injection preparation. The injection medium can be any pharmaceutically acceptable isotonic liquid. Examples include phosphate buffered saline (PBS), culture media such as DMEM (preferably serum-free), physiological saline or 5% dextrose in water. In cases having more in a range around the 20% tissue involvement severity level, multiple injections of rMSC are envisioned. Follow-up therapy may involve additional dosing regimens. In very severe cases, e.g., in a range around the 40% tissue involvement severity level, multiple equivalent doses for a more extended duration with long term (up to several months) maintenance dose aftercare may well be indicated.

The total amount of cells that are envisioned for use depend upon the desired effect, patient state, and the like, and may be determined by one skilled within the art. Dosages for any one patient depends upon many factors, including the patient's size, body surface area, age, the particular compound to be administered, sex, time and route of administration, general health, and other drugs being administered concurrently.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, cardiomyocytes, fibroblasts, muscle cells, or various stem or progenitor cells, in particular embryonic heart muscle cells, liver stem cells (International Patent Publication WO 94/08598), and the like. Preferably the cells utilized for gene therapy are autologous to the patient.

Apoptosis-Resistant rMSCs

Retrovirally transduced recombinant mesenchymal stem cells (rMSCs) that express genes whose products inhibit apoptosis or inflammation are specifically provided in the invention. When injected directly into infarcted regions of damaged hearts, these novel recombinant MSCs (rMSCs) resist cell death in the immediate peri-transplant period at least partly due to their ability to survive hypoxic conditions. Preferred anti-apoptotic genes protect against oxidative injury and are anti-inflammatory. Specifically contemplated anti-apoptotic candidate genes include the cytoprotective heme oxygenase (HO) gene, the serine-threonine kinase Akt (protein kinase B) gene and the extracellular superoxide dismutase (ecSOD) polypeptide; or a biologically active fragment, derivative, analog or homolog thereof. Additional recombinant nucleic acid molecules that encode cell survival proteins are known in the art and include, by non-limiting example, HIFA (hypoxia inducible factor), DEL-1 (developmental embryonic locus-1), NOS (nitric oxide synthase), BMP's (bone morphogenic proteins), P2-adrenergic receptor, and SERCA2a (sarcoplasmic reticulum calcium ATPase). In some embodiments, rMSCs are used as vectors for gene delivery to damaged tissue sites or diseased tissue sites in vivo. Grafted rMSCs are able to differentiate into cardiomyocytes and provide therapeutically meaningful improvements in cardiac function including reduced infarct volume, increased capillary density and function, and less overall scarring. Grafted rMSCs prevent post-injury tissue remodeling and restore normalized cardiac function (systolic and diastolic) after infarction.

Cardiac injury promotes tissue responses that enhance myogenesis using implanted rMSCs. Thus, rMSCs are introduced to the infarct zone to reduce the degree of scar formation and to augment ventricular function. New muscle is thereby created within an infarcted myocardial segment. Recombinant MSCs are directly infiltrated into the zone of infarcted tissue. The integration and subsequent differentiation of these cells is characterized, as described herein. Timing of intervention is designed to mimic the clinical setting where patients with acute myocardial infarction would first come to medical attention, receive first-line therapy, followed by stabilization, and then intervention with myocardial replacement therapy if necessary.

The severity of myocardial infarction to be treated, i.e. the percentage of muscle mass of the left ventricle that is involved can range from about 5 to about 40 percent. This includes affected tissue areas that one contiguous ischemia or the sum of smaller ischemic lesions, e.g., having horizontal affected areas from about 2 cm to about 6 cm² and a thickness of from 1-2 mm to 1-1.5 cm. The severity of the infarction is significantly affected by which vessel(s) is involved and how much time has passed before treatment intervention is begun.

The genetically engineered mesenchymal stem cells used in accordance with the invention, in order of preference, are autologous, allogeneic or xenogeneic, and the choice can largely depend on the urgency of the need for treatment. A patient presenting an imminently life threatening condition may be maintained on a heart/lung machine while sufficient numbers of autologous MSCs are cultured or initial treatment can be provided using other than autologous MSCs.

The proper environmental stimuli convert rMSCs into cardiac myocytes. Differentiation of rMSCs to the cardiac lineage is controlled by factors present in the cardiac environment. Exposure of rMSCs to a simulated cardiac environment directs these cells to cardiac differentiation as detected by expression of specific cardiac muscle lineage markers. Local chemical, electrical and mechanical environmental influences alter pluripotent rMSCs and convert the cells grafted into the heart into the cardiac lineage.

A series of specific treatments applicable to MSCs to induce expression of anti-apoptotic or cytoprotective genes are disclosed herein. Growth conditions for MSCs include those provided in Example 2 and those known in the art, e.g., as described in U.S. Pat. No. 6,387,369.

The rMSC therapy of the invention can be provided by several routes of administration, including the following. First, intracardiac muscle injection, which avoids the need for an open surgical procedure, can be used where the rMSCs are in an injectable liquid suspension preparation or where they are in a biocompatible medium which is injectable in liquid form and becomes semi-solid at the site of damaged myocardium. A conventional intracardiac syringe or a controllable arthroscopic delivery device can be used so long as the needle lumen or bore is of sufficient diameter (e.g., 30 gauge or larger) that shear forces will not damage the rMSCs. The injectable liquid suspension rMSC preparations can also be administered intravenously, either by continuous drip or as a bolus. During open surgical procedures involving direct physical access to the heart, all of the described forms of rMSC delivery preparations are available options.

Implantation of rMSCs in Cardiac Muscle

A strategy has been developed by which mesenchymal stem cells can be isolated and rapidly expanded in culture. Once adequate numbers of cells are reached in culture, these cells can be administered back to the patient from whom they were raised. This technique of autologous transfer prevents the need for immunosuppressive protocols. Furthermore, techniques for highly efficient genetic manipulation of these cells, whereby over 90% of cells are transduced with the gene of choice, were developed. Genetic engineering of MSCs to express anti-apoptotic genes has not been described prior to this invention. The advantages of this important modification has been that it allows one (in this set of experiments) to overcome the limitations of cell death in the immediate peri-transplant period. Cell death in the first 24 hours of transplantation into the myocardium has previously been an insurmountable problem. Reinecke et al. have demonstrated that nearly all donor adult rat cardiac myocytes are lost twenty-four hours after implantation into cryo-injured adult rat hearts. Zhang et al. and Muller-Ehmsen et al. have shown that 30-60% of rat neonatal cardiac myocytes do not survive implantation into cryoinjured or uninjured hearts respectively, and it is well recognized that fetal cardiac myocytes do not survive transplantation into infarcted hearts.

Non-recombinant bone marrow-derived cells are even more susceptible to peri-transplantation cell death. Toma et al. estimate that 99.56% of human bone marrow-derived cells die 4 days after transplantation into uninjured nude-mouse hearts. Early attempts at preventing donor cell loss by subjecting rat skeletal myoblasts to heat-shock prior to transplantation have met with very limited success. The disclosed data indicates that genetic modification of stem cells to resist cell death can completely regenerate cardiac myocytes that are lost after infarction, and by doing so, we can completely normalize cardiac function (systolic and diastolic) after infarction, such that at least 20%, 30%, 40%, 50%, 60%, 70%, 75%, 80%, 85%, 90%, 95%, 99% or 100% of cardiac function is restored. Likewise, at least 20%, 30%, 40%, 50%, 60%, 70%, 75%, 80%, 85%, 90%, 95%, 99% or 100% of cardiac myocytes in the damaged tissue is regenerated.

The market for such a discovery is the rapidly expanding market for patients who are postmyocardial infarction, with symptomatic (or unsymptomatic) left ventricular dysfunction, and those with congestive heart failure. This is a rapidly growing market. The number of hospitalizations for congestive heart failure (CHF) alone increased from 377,000 in 1979 to 978,000 in 1999; and deaths due to CHF alone increased by 157% in the same period. This disease exacts a heavy toll on the American health care system. It is estimated that approximately 4.9 million Americans now carry the diagnosis of CHF, and 400,000 new cases occur annually. From a financial perspective, costs related to heart failure comprise $20.3 billion in direct costs and $2.2 billion in indirect costs, for a total of $22.5 billion a year.

Genetically modifying stem cells prior to implantation is not limited to manipulation of these cells for “anti-death” strategies, but includes genetic engineering to: (i) secrete angiogenic growth factors; (ii) overcome immunologic differences; (iii) control MSC proliferation; (iv) enhance MSC homing to ischemic myocardium; (v) enhance MSC engraftment in ischemic myocardium; and (vi) enhance contractile function after engraftment.

The present invention is further illustrated, but not limited, by the following example.

### EXAMPLES

### Example 1

### Purified Bone-Marrow Derived Mesenchymal Stem Cells

Prolonged interruption of myocardial blood flow initiates events that culminate in the death of cardiac myocytes. Endogenous reparative mechanisms such as cardiac myocyte hypertrophy and hyperplasia; and trafficking of bone marrow-derived cells to the myocardium for purposes of angiogenesis and myogenesis are capable of restoring only a miniscule portion of lost myocardial volume, and have little functional impact. Attempts to recruit these reparative mechanisms for therapeutic purposes, for example, by mobilizing bone marrow-derived stem cells before, during and after experimental myocardial infarction (MI) using systemic administration of granulocyte colony-stimulating factor (G-CSF) have failed to fully restore lost myocardial volume or to normalize cardiac function. Other groups have harvesting bone marrow-derived cells for injection into ischemic myocardium and demonstrated that this results in both angiogenesis and myogenesis, with incomplete replacement of lost myocardium, and modest functional improvement. Because it is now known that cells with exclusive angiogenic potential are mobilized from bone marrow and home to the ischemic myocardium where they induce vasculogenesis and angiogenesis, it is unclear how much of the functional improvement reported in the above studies is due to the protective effects of angiogenesis on native cardiac myocytes, and how much is due to true myogenesis. Furthermore because isolating adequate numbers of pure populations of cells capable of myogenesis has proven to be technically challenging, it is unlikely that strategies reported to date will find meaningful clinical translation.

Mesenchymal stem cells are self-renewing, clonal precursors of non-hematopoietic tissues. They are expandable in culture, multi-potent and can differentiate into osteoblasts, chondrocytes, astrocytes, neurons and skeletal muscle. The group from Osiris Therapeutics has reported that putative MSCs derived from bone marrow that express C090 and proprietary markers SH-2 and SH-3, but not CD117 (c-kit) can differentiate into cardiac muscle in vivo However, implantation of as many as 6×10⁷ MSCs into infarcted porcine hearts yielded no improvement in cardiac function, because an estimated >99% of human bone marrow-derived MSCs die four days after transplantation into uninjured nude-mouse hearts.

Although conceptually attractive, cell transplantation strategies to replace lost myocardium are limited by the inability to deliver large numbers of cells that resist pert-transplantation death to the ischemic myocardium. Reinecke et al. have demonstrated that nearly all donor adult rat cardiac myocytes are lost 24 hours after implantation into cry-injured adult rat hearts. Zhang et al. and Muller-Ehmsen et al. have shown that 30-60% of rat neonatal cardiac myocytes do not survive implantation into cryo-injured or uninjured hearts respectively; and fetal cardiac myocytes do not survive transplantation into infarcted hearts. Early attempts at preventing donor cell death have met with limited success.

Accordingly, a pure population of adult rat bone marrow-derived MSCs were isolated, characterized and expanded. Then the cells were tested to determine whether they differentiate into cardiac myocytes in vivo and participate in cardiac repair after transplantation into the ischemic rat heart. Since regenerative capacity is limited by cell death in the peri-transplantation period, we engineered MSCs to over-express Akt prior to transplantation. This serine-threonine kinase is a powerful survival signal in many systems and exerts its anti-apoptotic effects at least in part, by inactivation of Bad and caspase-9, and by activation of pro-survival molecules Bcl-2 and IKK. Using this strategy, retention of greater numbers of MSCs in the ischemic myocardium translated into greater volume of regenerated myocardium after 3 weeks, normalization of systolic and diastolic cardiac function, and prevention of remodeling.

In this study, the strategy was to isolate a population of highly purified bone-marrow derived mesenchymal stem cells (MSCs) and to employ genetic engineering to render these cells resistant to apoptosis. The mononuclear fraction of whole bone marrow from adult Sprague-Dawley rats was isolated, and MSCs were isolated and purified. These c-kit+ CD34-cells did not differentiate into cells of hematopoietic lineage. Cells were stably transduced to over-express an Akt protein that was activated in the presence of hypoxia and serum-starvation, and protected MSCs from apoptosis in vitro. Upon transplantation into ischemic myocardium, 5 million Akt-MSCs were largely resistant to apoptosis in the peri-transplantation period, and differentiated into cardiomyocytes in vivo. Three weeks after experimental infarction and implantation of cells volume of regenerated myocardium was 3 to 4-fold higher than when equivalent numbers of GFP-transduced MSCs (5×10e6) were transplanted. These differences translated into significant systolic and diastolic functional improvement on isolated Langendorff preparations. In conclusion, bone marrow-derived MSCs capable of cardiomyogenesis can be isolated, purified and expanded in culture. Akt gene transfer of MSCs resulted in significant decrease in cell death, increases of volume of regenerated myocardium, and improvement in myocardial function. Such a customizable cell-based gene therapy strategy offers a potential solution to scalability issues that hinder effective and safe human translation of cell therapy for diseases of the myocardium.

Several groups have reported the use of un-fractionated or cell sorted bone marrow derived cells for cardiac repair. The characterization, expansion and conditions for differentiation of these cells need further definition. A pure subpopulation of CD34-/c-kit+ adult rat bone marrow-derived MSCs were isolated, characterized, and propagated. This subpopulation of CD34-/c-kit+ mesenchymal stem cells can differentiate into cardiac myocytes and be transduced to stably express a reporter gene. These cells can induce a gain of cardiac function when transplanted into the myocardium damaged by ischemic injury.

The mononuclear fraction of whole bone marrow from adult Sprague Dawley rats was separated by density centrifugation. Bone marrow stromal cells attached preferentially to uncoated plastic surfaces, and proliferated in mixed culture with hematopoietic cells (HCs) under standard conditions. MSCs were retrovirally transduced with green fluorescent protein (GFP) or Lac Z with over 80% transduction efficiency. MSCs express connexin 43 and c-kit (CD117) but do not express hematopoietic markers CD34, CD45, CD11b; or mature cardiac markers such as troponin, myosin heavy chain or desmin at this stage. MSCs can be separated from HCs by negative immuno-magnetic bead sorting, but cease to proliferate after cell sorting. Lac Z transduced MSCs from a male donor rat were injected into the border zone of the ischemic myocardium 60 minutes after ligation of the female rat LAD. Two weeks later, the free wall and apex of the left ventricle exhibited extensive blue staining by beta-galactosidase staining, indicating the presence of Lac-Z expressing cells. The transgene and &-chromosome co-localized with markers of mature cardiomyocytes, myosin heavy and light chains, alpha sarcomeric actin and cardiac troponin. Echocardiographic analysis revealed a statistically significant 54% increase in fractional shortening when compared to control, and a 34% increase in ejection fraction.

The data reported herein indicates that bone marrow derived MSCs can be expanded to sufficient scale ex vivo, and genetically engineered to successfully restore the function of damaged myocardium.

The methods are useful for ex-vivo expansion of stem cells in order to reach clinically useful amounts, autologous transfer, and genetic modification of autologous stem cells to enhance function prior to transfer back into patient. This invention therefore includes method of isolation, culture, and purification of bone marrow-derived mesenchymal stem cells and markers of mesenchymal stem cells isolated as described in Example 2. Also included are techniques for reporter gene and therapeutic gene transfer and demonstration that MSCs isolated in this fashion differentiate into cardiac myocytes. Evidence that therapeutic gene transfer results in a significant improvement in end-points include increased survival of rMSCs, increased volume of regenerated myocardium, and increased cardiac function, when compared to mesenchymal stem cell transplantation alone.

### Example 2

### Isolation, Genetic Engineering, and Increased Function of rMSCs

1. Isolation, Culture and Purification of Bone Marrow Derived Mesenchymal Stem Cells.

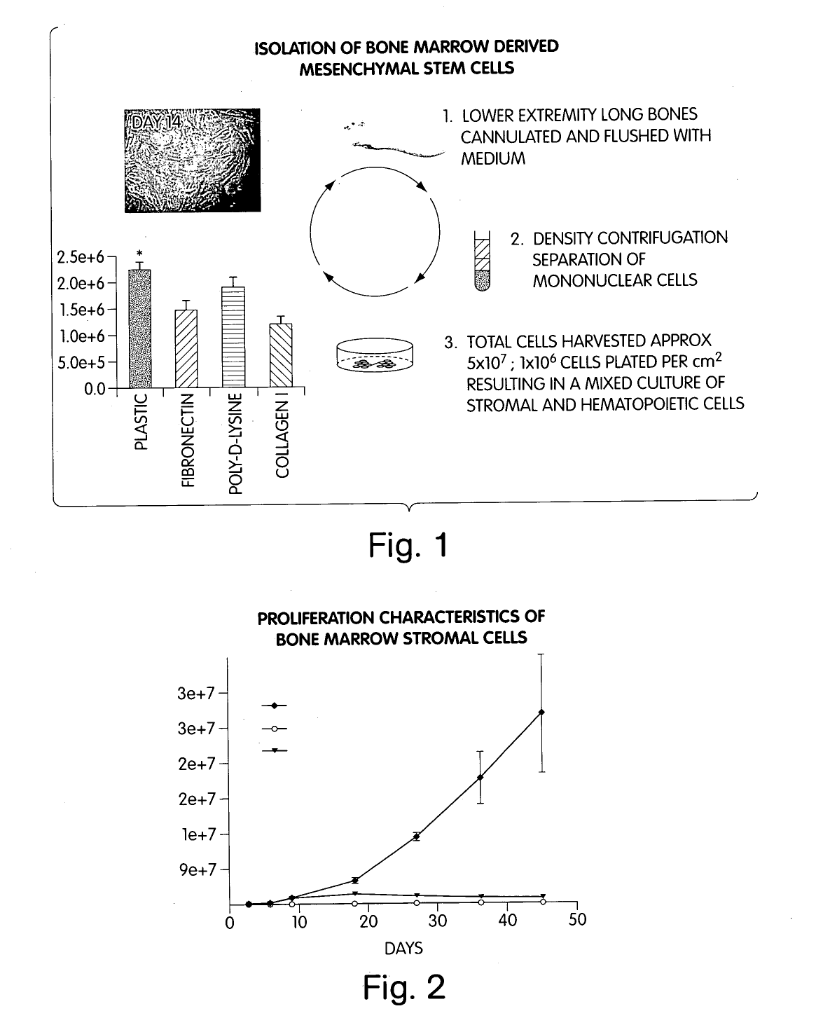

Adult mate Sprague-Dawley rats (200 grams) were purchased from Harlan Laboratories (Indianapolis, Ind.). The animals were maintained on a 12:12 light:dark cycle at an ambient temperature of 24° C. and 60% humidity. Food and water were provided ad libitum. They were anesthetized using intraperitoneal ketamine (70 mg/kg) and xylazine (4 mg/kg). The tibia and femur of both lower extremities were harvested using sterile surgical technique, and then cannulated at the epiphyseal plate with a 21-gauge needle. The marrow cavity was flushed three times with 30 mL of complete medium. 13 mL of Ficoll 1.077 solution (Pharmacia, Peapack, N.J.) was layered under the cell suspension, and centrifuged for 20 minutes at room temperature. The buffy coat was harvested, washed twice with phosphate-buffered saline, and then counted using a standard hematocytometer. A total of approximately 5×10⁷ cells were harvested from each animal. Of these, 1×10⁶ cells were plated per cm on 55 cm² polystyrene cell culture plates (Corning, and attached preferentially to the polystyrene surfaces when compared to collagen, retronectin or poly-D-lysine coated plates. A flow diagram is provided in FIG. 1.

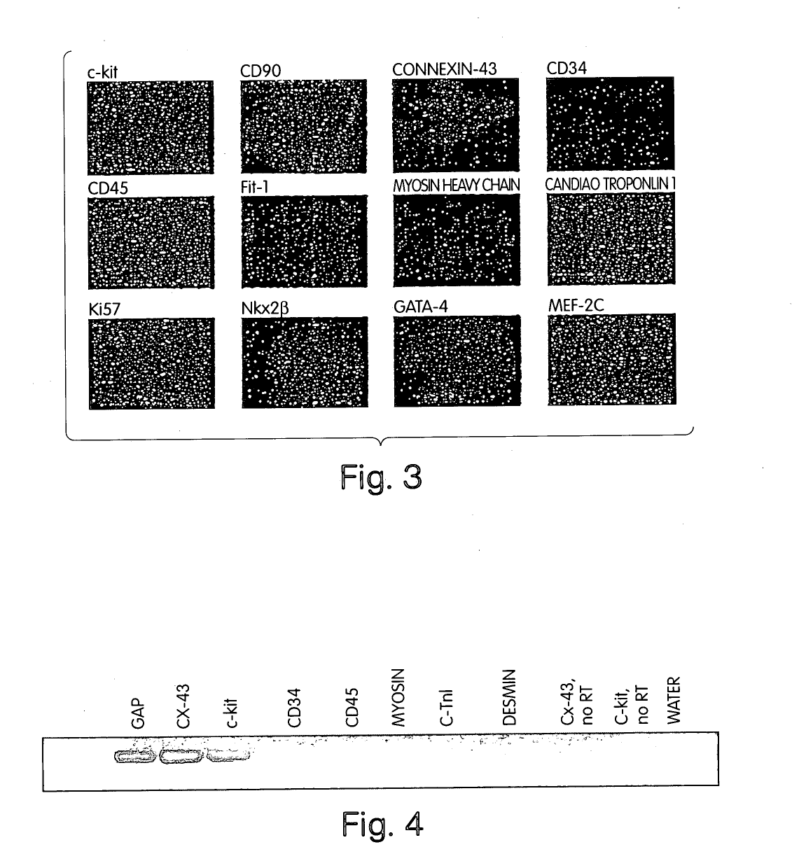

Cells were cultured at 37° C. in 5% CO₂, in complete medium, which consisted of Alpha Minimal Essential Medium (Invitrogen. Carlsbad, Calif.) supplemented with lot-selected 20% fetal bovine serum (Invitrogen, Carlsbad, Calif.), antibiotic and anti-mycotic solution (Invitrogen, Carlsbad, Calif.) and 2 mM glutamine (Invitrogen, Carlsbad, Calif.). The first medium change was performed on Day 3. Cells were passaged by treating lightly with 0.025% Trypsinl/0.01% EDTA in HBSS (Clonetics, Walkersville, Md.) and counted every three days from day 3, to day 48. Cells proliferated rapidly in culture as shown in the graph on the right, yielding 2.5×10⁵ cells by day 9 and 5×10⁵ by day 15 of culture. MSCs from bone-marrow of adult male rats attached to uncoated plastic surfaces. Hematopoietic cells failed to attach and were removed with medium changes. Proliferation characteristics of isolated MSCs grown in culture are provided in FIG. 2.

2. Markers of Mesenchymal Stem Cells Isolated by Technique Described Above

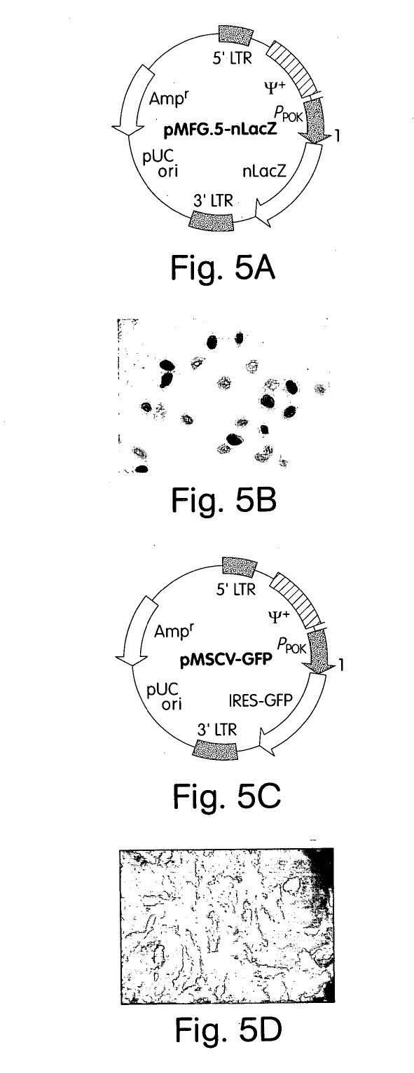

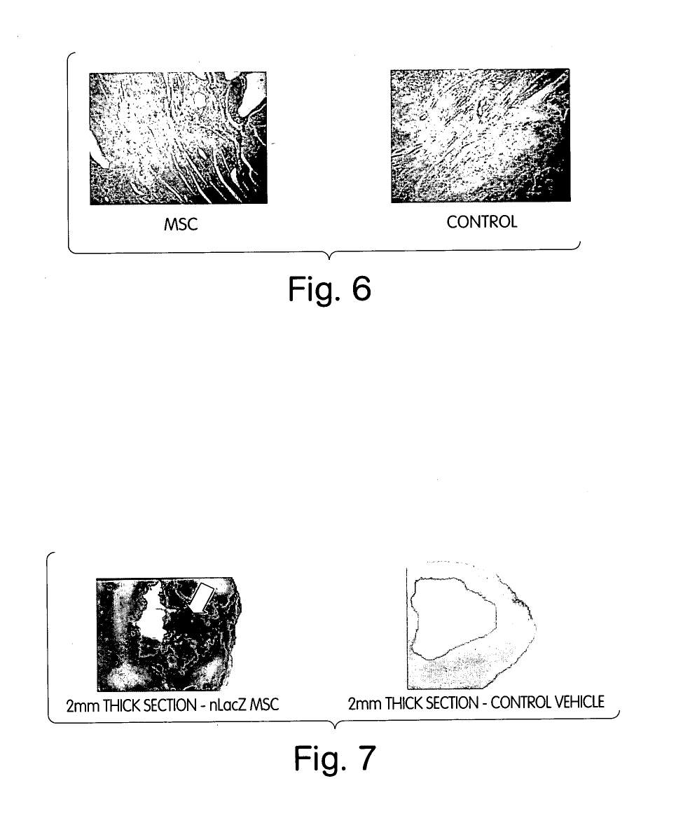

MSCs were tested for expression of stem cell markers that are distinct from hematopoietic stem cells. On immunocytochemistry, over 99% of MSCs expressed connexin-43, c-kit (CD117) and CD90, 60% expressed Ki67, and 15% expressed Nkx2.5, and GATA-4. MSCs did not express CD34, CD45, myosin heavy chain (MHC), myosin light chain (MLC), cardiac troponin I (CTnI), alpha-sarcomeric-actin (α-SA), or cardiac-specific transcription factor MEF-2. See, FIG. 3. These observations were verified by RT-PCR. See, FIG. 4. Cell surface marker expression were found to be quite different from that described by others (e.g., Osiris Therapeutics). For example, Osiris Therapeutics do not report expression by MSCs of CD 117 (c-kit) but do report expression of CD90 and propriety markers SH-2 and SH-3. Determining expression markers allowed development of a negative paramagnetic bead sorting method targeting CD34 in order to obtain a >99.9% pure MSC population. In order to do this, avidin coated magnetic beads (Beckman Coulter, Fullerton, Calif.) were linked with monoclonal antibodies to rat CD34 (BD Pharmingen, Franklin Lakes, N.J.) that had been biotinylated (Sigma. St. Louis, Mo.) at 4° C. overnight. This preparation was then incubated with cells suspended in 30% FBS for 30 minutes at RT and then exposed to a magnet for 20 minutes. The clear supernatant was harvested, and the procedure repeated once. The cells were then harvested and resuspended in complete medium.

Attempts were made to induce MSCs to differentiate into megakaryocytes and erythroid cells per published protocols. An inability to do so would suggest that these cells were indeed distinct from hematopoietic stem cells. In order to demonstrate differentiation into megakaryocytes, complete medium was supplemented with 100 IU/mL Thrombopoietin+80 IU/mL IL-3+80 IU/mL GM-CSF+2 IU/mL c-kit ligand using standard methods and MSCs maintained in culture for 14 days. Staining was then performed for megakaryocyte markers CD61 and CD42a in order to demonstrate differentiation into erythroid elements, complete medium was supplemented with 2 IU/mL Erythropoietin+100 IU/mL thrombopoietin+80 IU/mL IL-3+80 IU/mL GM-CSF+2 IU/mL c-kit ligand. Staining was then performed for erythrocyte marker TR-1119 as previously reported. As expected, these attempts were unsuccessful indicating that MSCs are distinct from cells of hematopoietic lineage. Attempts were also made to induce MSCs to differentiate into cardiac myocytes in vitro using varying concentrations of 5-azacytidine, per published protocols but were also unsuccessful.

3. Genetic Modification of Mesenchymal Stem Cells Using Retroviral Transduction.

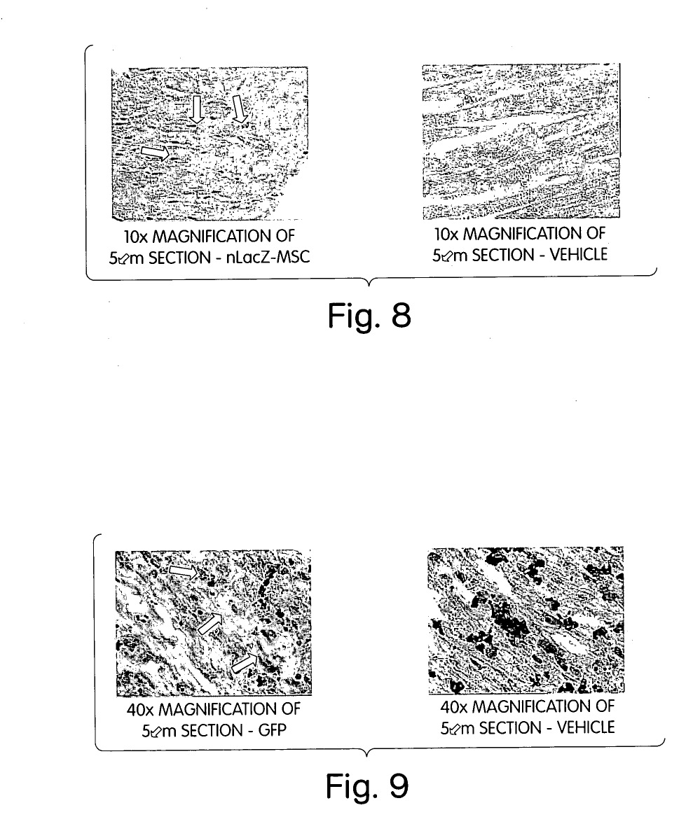

The Murine Stem Cell Virus Vector (Clontech, Palo Alto, Calif.) was obtained and digested with XhoI and Bam HI. IRES-GFP was then cloned into these sites. See, FIG. 5. A cDNA encoding a constitutively active murine Akt was cloned into the Murine Stem Cell Virus Vector. Akt was PCR-amplified using primers 5′-GCAAGATCTG ATACCATGAA CGACGTAGCC-3′ (SEQ ID NO:1) and 5′CGGTCACCGT GTCGGACTCC TAGGATC-3′ (SEQ ID NO:2), and cloned into pMSCV using Bgl II and BamHI. Plasmids expressing nuclear localized LacZ (nLacZ) and high titer VSV-G pseudotyped retroviruses were generated separately by tripartite transfection of 293T cells and concentrated by ultracentrifuge. Southern blot analysis on infected 3T3 cells yielded titers of approximately 5×10⁸ viral particles per mL. Retroviral supernatant was then aliquoted and stored at −80° C. MSCs were exposed to 1×10⁸ particles with 6 μg/mL polybrene (Sigma-Aldrich, St. Louis, Mo.) for 6 hours, after which medium was replaced. 18 hours later, transduction was repeated. Three cycles were performed 7 to 9 days after harvest. First-passage cells were used for intramyocardial injection 4-5 days after the last transduction. Transduction efficiency was assessed by ultraviolet examination and immunohistochemistry for GFP, X-gal staining for nLacZ gene transfer, and by Western blot for Akt.

4. MSCs Isolated as Above Differentiate into Cardiac Myocytes after Transplantation into the Ischemic Heart

Adult female Sprague-Dawley rats (300-350 grams) were purchased from Harlan Laboratories (Indianapolis, Ind.) and maintained as previously described. After the induction of anesthesia, the animal was intubated and mechanically ventilated (Harvard Rodent Ventilator, Harvard, Mass.). A left thoracotomy was performed in the fourth inter-space and the heart exposed. The proximal left anterior descending (LAD) artery was identified and ligated using 7-0 prolene suture (Ethicon, Somerville, N.J.). The animal was maintained at a surgical plane of anesthesia for 60 minutes with the chest open. Varying amounts of transduced MSCs suspended in 250 μL normal saline (n=6), or saline, or control-cells for the control animal (n=6 for GFP, n=6 for LacZ) were injected sub-epicardially with an angled 27-gauge needle into five sites in the anterior and posterior left ventricle in the border-zone between ischemic and normal myocardium. This border zone was evident to the naked eye. After injection, the heart was observed for several minutes. Once normal sinus rhythm and hemostasis was obtained, the chest was closed in layers with 3-0 and 4-0 nylon (Ethicon, Somerville, N.J.) and the animals were allowed to recover. Hearts were excised 24 hours, 72 hours and 3 weeks after injection. Area at risk was estimated by Evans blue retrograde perfusion, and expressed in arbitrary units. The left ventricle was sliced into eight transverse slices of equal thickness from apex to base. One group of thick slices was fixed in gluteraldehyde, stained for beta-galactosidase (Invitrogen, Carlsbad, Calif.), frozen in OCT compound and sectioned at 5 μm. All other thick slices were fixed in formaldehyde, paraffin-embedded and sectioned at 5 μm. Hemotoxylin and eosin (H&E) and Masson's trichrome staining was done. Left ventricular volume was calculated by dividing weight by density (1.06 gm/mL). On Masson's trichrome stain, the blue to non-white ratio of surface area was calculated for twenty 5 μm sections from each thick slice, and multiplied by the thickness of the whole left ventricle to calculate volume of infarct. Volume of viable myocardium was calculated by subtracting infarct volume from total LV volume. Volume of regenerated myocardium was calculated by subtracting volume of viable myocardium in control animals from that in all other experimental groups Collagen area fraction and cardiomyocyte surface area were assessed as previously described. Separately, sections were incubated with primary mAb to c-kit, GFP, Ki-67, cardiac troponin, myosin heavy chain, myosin light chain, N-cadherin, and connexin-43 (Sigma-Aldrich). Appropriate fluorochrome-linked secondary mAbs were used and slides were visualized. Fluorescent in-situ hybridization was performed using green fluorescent Y chromosome enumerator hybridization probes with commercially available kits (Vysis, Downers Grove, Ill.). Sections were counterstained with Hoechst 33258.

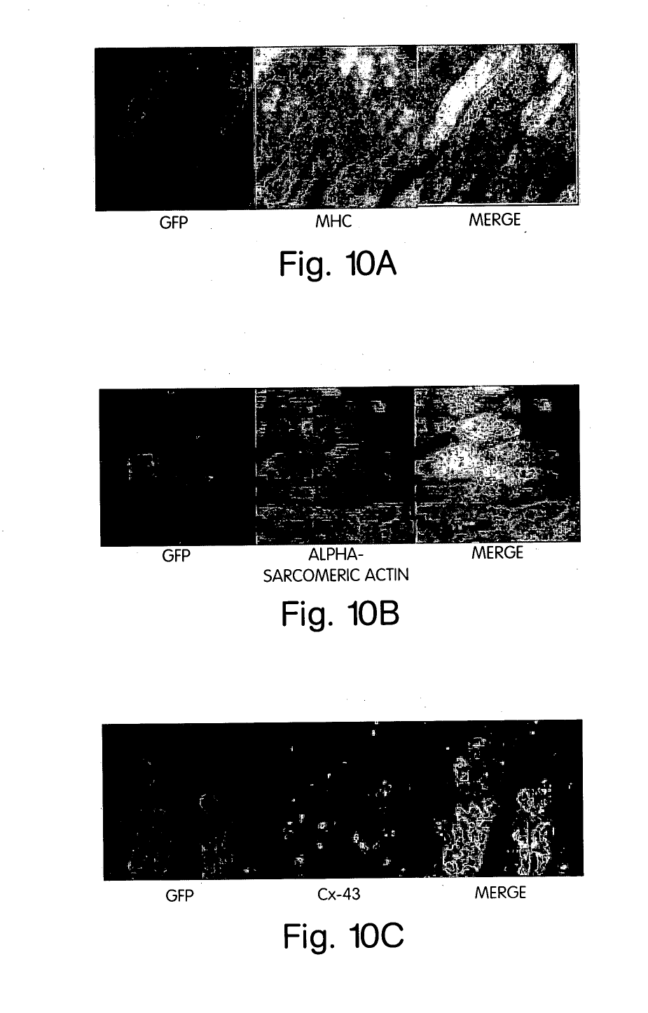

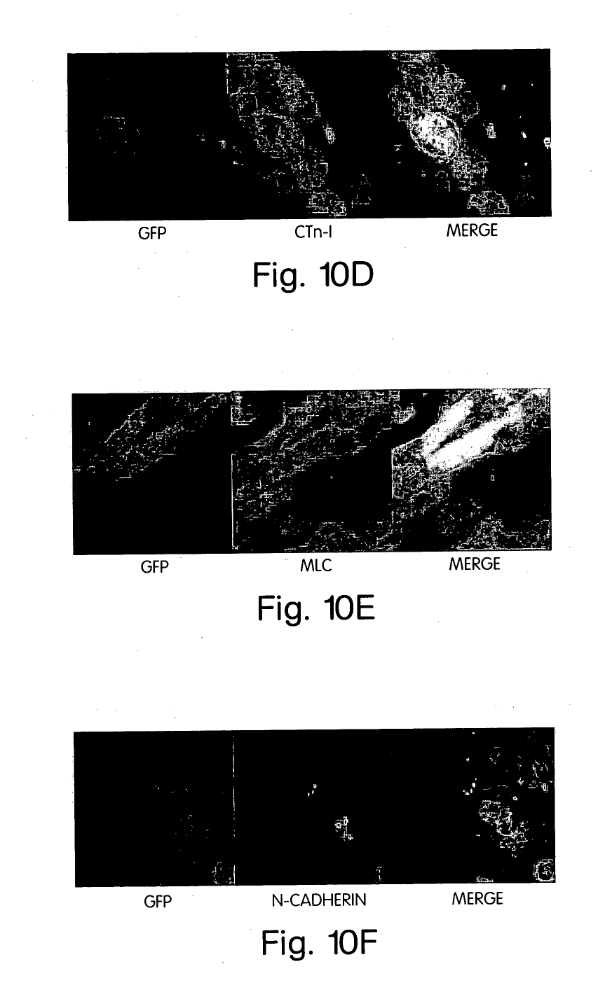

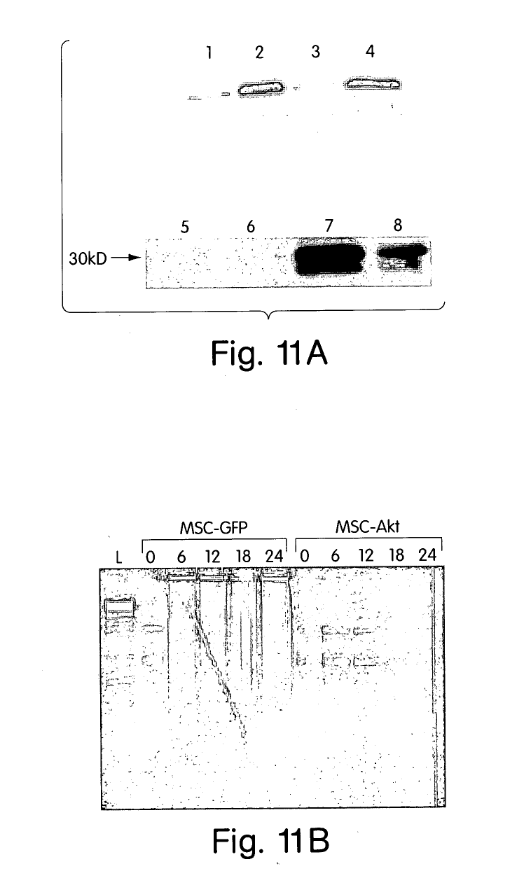

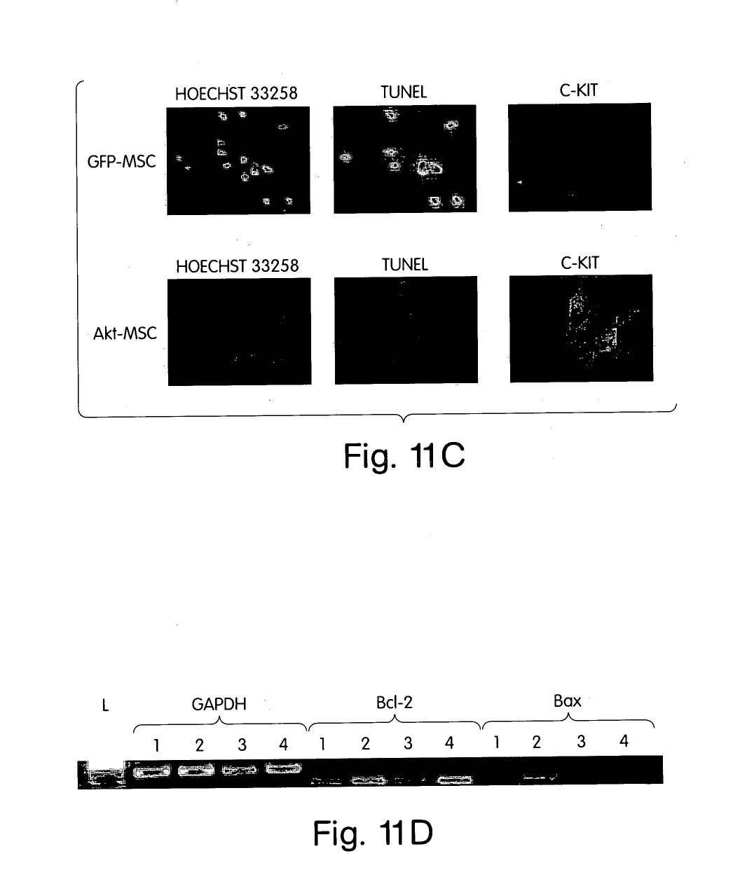

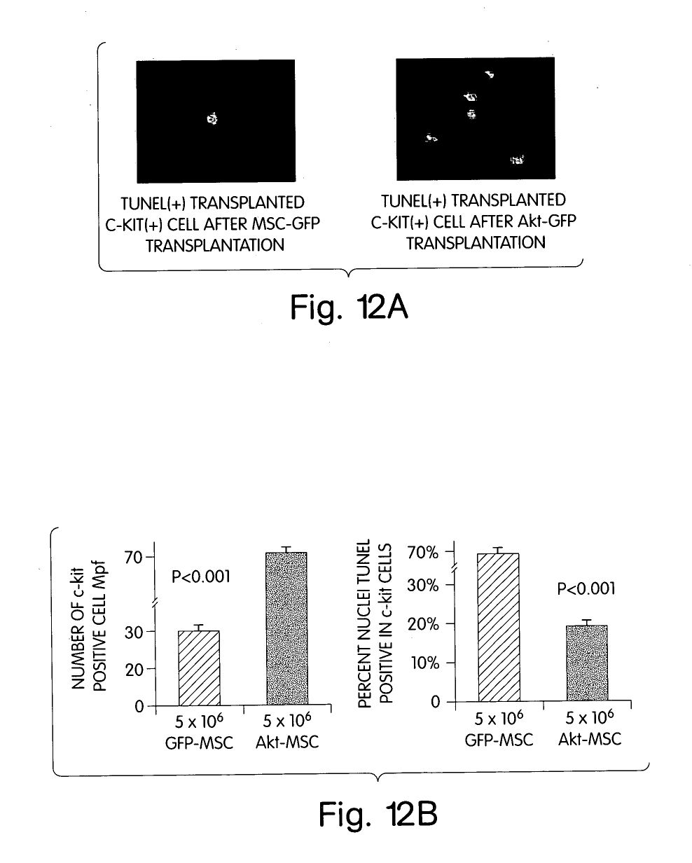

On H&E staining of myocardium after infarction and MSC injection, the residual scar was infiltrated by finger-like extensions of organized cardiac myocytes. On β-galactosidase staining of whole heart thick sections injected with 5×10⁵ LacZ-MSCs, we observed intense blue coloration in the peri-infarct zone that was due to blue nuclear staining of cells that bore the phenotype of cardiac myocytes. See, FIGS. 6-9. When ischemic hearts injected with GFP-MSCs were stained for GFP, large, multi-nucleated syncitiae, oriented in the same direction as the native cardiac myocytes, were observed in the border zone. See, FIGS. 6-9. Experiments were carried out to verify that cells expressing the transgene also expressed cardiac specific markers. See, FIG. 10. Sections were double-stained for GFP and cardiac-specific proteins. GFP co-localized with MHC, MLC, CTnI, desmin and α-SA. Regenerated cardiac myocytes expressed connexin-43 and N-cadherin at contact points with native cardiac myocytes, indicating the capacity for electro-mechanical coupling. The donor origin of regenerated cardiac myocytes was verified by fluorescent in-situ hybridization for the Y-chromosome, which co-localized with the above-mentioned cardiac-specific proteins. Cardiac myocytes expressing the transgene and/or Y-chromosome did not express c-kit or CD90 three weeks after transplantation. Cardiac myocytes expressing the transgene were not identified after injection of MSCs into uninjured myocardium. The transgene was not identified in endothelium, smooth muscle or hematopoietic elements within the ischemic heart. After injection into the border zone, cardiac myocytes expressing the transgene were not found in remote areas of the heart (e.g. right ventricle or atria). Cardiac myocytes expressing the transgene after injection of c-kit/CD34⁺ cells into ischemic myocardium were not identified. No ectopic tissue or tumors were identified within the myocardium after MSC injection.

5. Demonstrate that Therapeutic Gene Transfer of Akt to MSCs Results in a Meaningful Improvement in End-Points—rMSC Survival, Volume of Myocardium Regenerated and Cardiac Function.

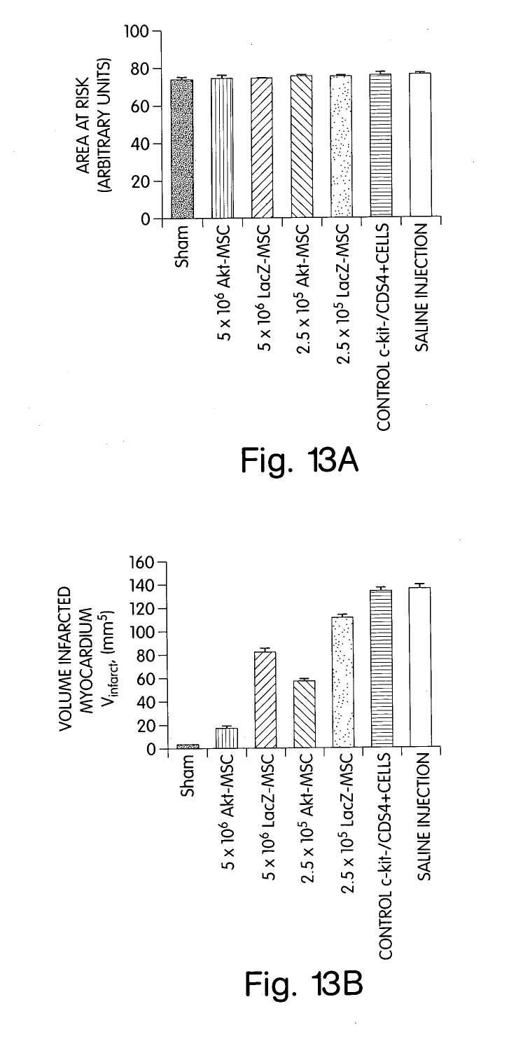

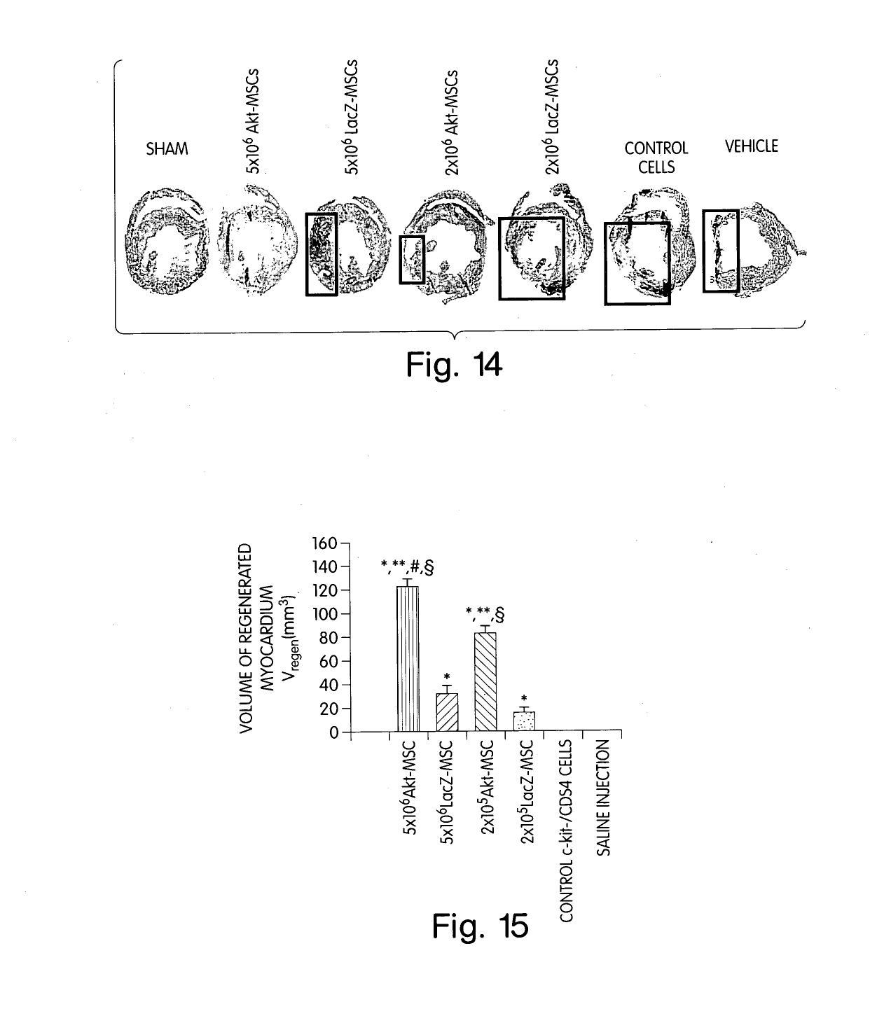

In order to test whether Akt-gene transfer to MSCs enhanced survival in vitro, a series of simulated hypoxia-reoxygenation protocols were generated. Fourteen days after successful retroviral gene transfer, and induction of differentiation into cardiomyocytes, cells were subjected to a simulated hypoxia-reoxygenation protocol. Compete medium was replaced with serum free medium, and cells placed in a hypoxia chamber (Coy Laboratory Products, Grass Lake, Mich.) with 1% ambient oxygen at 37 C for 0, 6, 12, 18 and 24 hours. Cells were then moved to 21% ambient oxygen at 37 C, and medium was replaced with complete medium. Ten minutes into the “reoxygenation phase,” protein was extracted for Akt assay which tested the ability of Akt immunoprecipitated from the lysate to phosphorylate 1 microgram of GSK-3 fusion protein. 24 hours after “reoxygenation” DNA was harvested for ladder, RNA was harvested for RT-PCR, and TUNEL assay to assess apoptosis was performed. See, FIGS. 11-12.

Increased Akt activity protected against MSC apoptosis in vitro and in vivo. At baseline conditions of 37° C. and 21% ambient oxygen, Akt activity was equivalent in both groups. After 24 hours of hypoxia in sewn-free medium, Akt activity increased 28.5-fold in the Akt-MSC group, and 6.6-fold in the GFP-MSC group reducing MSC apoptosis by 79%, and reducing DNA laddering. The protective effects of Akt in vivo was assessed by double-staining left ventricular sections for c-kit and TUNEL, allowing determination of the number of c-kit⁺ cells retained in the myocardium, and the percent of c-kit⁺ cells that were apoptotic. Twenty-four hours after transplantation of 5×10⁵ LacZ-MSCS into ischemic myocardium, 68% of 33±1.53 LacZ-MSCs per high power field (hpf) were apoptotic. By contrast, twenty four hours after transplantation of 5×10⁵ Akt-MSCs only 19% of 82±6.7 Akt-MSCs per hpf were apoptotic (p<0.001)). An additional forty-eight hours later, 31% of 22.7±9.8 LacZ-MSCs per hpf were apoptotic; whereas 17% of 66±3.5 Akt-MSCs per hpf were apoptotic (p<0.001). There were no c-kit+ cells present in the myocardium after three weeks. These observations indicate that Akt was activated in MSCs exposed to hypoxia and serum-starvation in vitro, as well as after transplantation into the ischemic myocardium, and that increased Akt activity prevents MSCs apoptosis in the immediate post-implantation period. See, FIGS. 11-12.

Intramyocardial rMSC Injection Reduces Infarct Volume

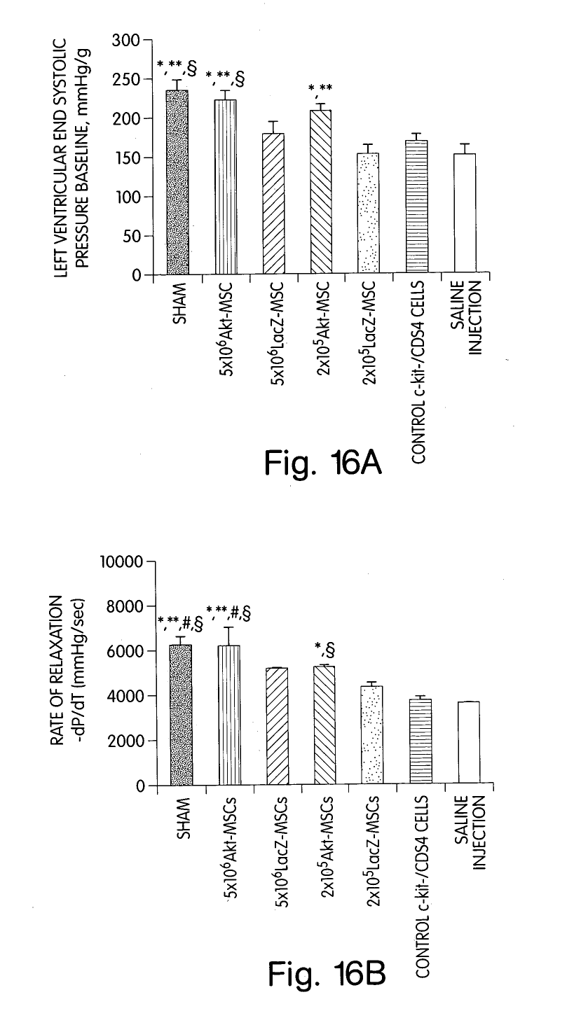

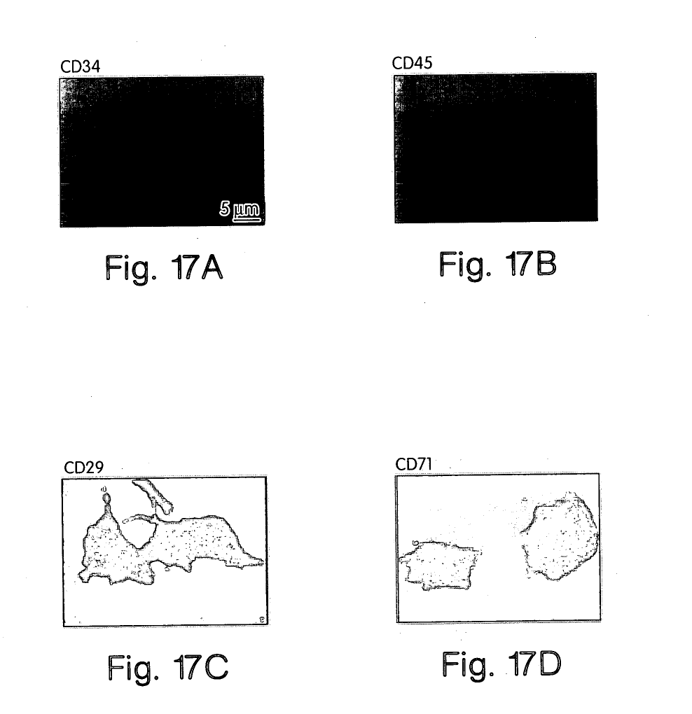

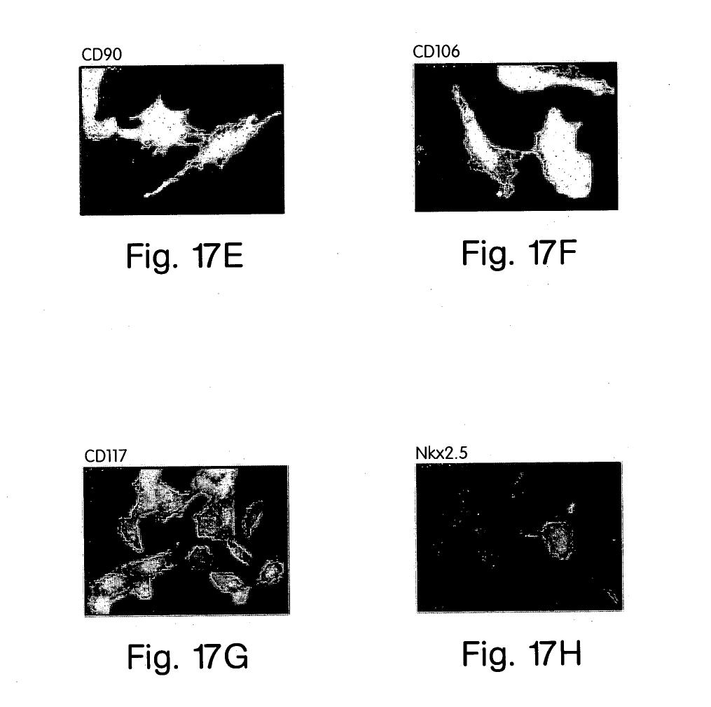

Peri-operative mortality was approximately 12.5% in all groups due to tamponade. There were no late deaths in any group. The area at risk after coronary artery ligation was equivalent in all groups. Three weeks after ligation, the volume of left ventricular infarct (V_{infarct}) varied based on type and number of cells injected. V_{infarct} was greatest after saline injection and injection of control c-kit/CD34⁺ cells. V_{infarct} decreased after MSC injection in dose-dependent fashion. Injection of 2.5×10⁵ LacZ-MSCs yielded a 9.8% reduction in V_{infarct} and injection of 5×10⁶ LacZ-MSCs yielded a 12.9% reduction in V_{infarct}. Genetic modification with Akt exerted a more powerful effect on V_{infarct}, as V_{infarct} decreased by 44.8% after injection of 2.5×10⁶ Akt-MSCs. Combining a large starting cell number with Akt modification with injection of 5×10⁶ Akt-MSCs resulted in almost complete obliteration of V_{infarct}. See, FIGS. 13-15. This reduction was almost entirely due to an increase in volume of regenerated myocardium (V_{regen}). Intramyocardial injection of 5×10⁶ Akt-MSCs resulted in regeneration of 84.7% of lost myocardium. Administration of 2.5×10⁵ Akt-MSCs resulted in 2.5-fold more (V_{regen}) than administration of 20-fold more LacZ-MSCs. Enhanced MSC viability in the immediate peri-transplant period, resulting in greater (V_{regen}) and therefore, smaller V_{infarct}, was observed. Capillary density in the border zone was similar in sham-operated animals as well as in all groups receiving cell transplantation, but exceeded that in the control groups. See, FIGS. 13-15.

Akt-MSC Transplantation Normalizes Cardiac Function

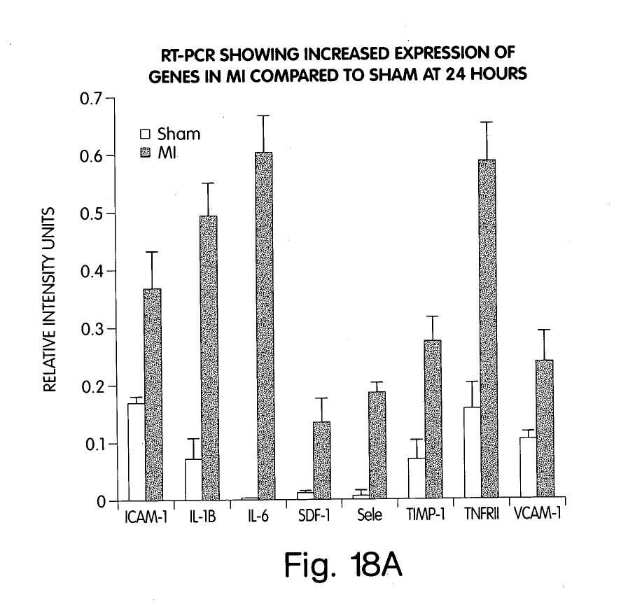

In order to assess cardiac function, hearts of rats were isolated and retrogradely perfused in the Langendorff mode. Isovolumic contractile performance was measured by placing a polyvinylchloride balloon in the left ventricle connected to a data acquisition system at baseline, in the presence of saturating concentrations of dobutamine, and after dobutamine washout, as previously described Left ventricular end-systolic pressure (LVESP) and end-diastolic pressure (LVEDP), left ventricular developed pressure (LVDP), rate pressure product (RPP), and rate of contraction and relaxation (±dP/dT) were measured. See, FIG. 16. Echocardiography was performed as previously described. Transplantation of 2.5×10⁵ LacZ-MSCs or 5×10⁶ LacZ-MSCs did not improve left ventricular systolic performance when compared to control cell or saline injection. Transplantation of 2.5×10⁵ Akt-MSCs resulted in statistically significant, 37% increase in baseline LVESP. In dose-dependent fashion, transplantation of 5×10⁶ Akt-MSCs resulted in a 50% increase in baseline LVESP, which was indistinguishable from LVESP of sham-operated animals. These differences persisted during dobutamine challenge, and were similar to those seen for left ventricular developed pressure, rate pressure product, and +dP/dT. After dobutamine challenge, rate of relaxation (−dP/dT) was slowest in control infarct and control cell injected animals. Injection of 2.5×10⁵ LacZ-MSCs or 5×10⁶ LacZ-MSCs yielded statistically insignificant 8% and 18% increases in −dP/dT, respectively. However, injection of 2.5×10⁵ Akt-MSCs resulted in a significant 22% increase in −dP/dT, and injection of 5×10⁶ Akt-MSCs resulted in 50% increase in −dP/dT which was equivalent to that in a sham-operated animal. Normalization of ejection fraction and fractional shortening on echocardiographic assessment was observed in sedated, conscious animals after injection of 5×10⁶ Akt-MSCs. See, FIG. 16.

Akt-MSC Transplantation Prevents Remodeling

Transplantation of 5×10⁶ Akt-MSCs reduced cardiac CD45⁺ cell infiltration by 67%, to the level encountered in sham-operated animals. Similarly, transplantation of 5×10⁶ Akt-MSCs reduced whole heart collagen-area fraction by 89.6%, and native cardiac myocyte surface area by 81%. All three parameters were comparable to levels observed in sham-operated animals. Transplantation of 5×10⁶ LacZ-MSCs did not achieve such a magnitude of reduction, and was statistically insignificant when compared to control animals. These observations all indicate that improved retention of MSCs in the peri-transplantation period, with subsequent high levels of engraftment and differentiation as described herein, exerts a powerful protective effect on cardiac remodeling.

### Example 3

### General Methods

Data was generated using the following reagents and methods.

Plasmids and hHO-1 Vector Construction

A 986 bp fragment of hHO-1 containing the open reading frame sequence was cleaved from the pBS KS (−) cloning vector at KpnI-PstI sites and subcloned at the corresponding sites in pUC 18 plasmid. The insert was cut at EcoRI sites and cloned into corresponding sites in an adeno-associated viral backbone (pAAV_{CMV-HO-1}) containing the human cytomegalovirus (CMV) immediate early gene promoter and the bovine growth hormone polyadenylation signal flanked by the AAV inverted terminal repeats encoding the required replication and packaging signals. Packaging, propagation and purification of AAV viral particles was carried out using standard procedures.

rAAV Production and Infection:

Recombinant AAV (rAAV) were produced in our Viral Core Facility by using the tHREe plasmid cotransfection system. Briefly, HEK293 cells were grown in MEM containing 10% FBS. To generate AAV virus, the cells were cotransfected with 17 μg of transgene plasmid per dish along with 17 pg of plasmid pHLPI9 and 17 pg of plasmid pLadeno5 per dish. PHLPI9 has AAV rep and cap genes, which provide the trans functions of rAAV. Adeno5 has the adenoviral VA, E2A and E4 regions that mediate rAAV replication. The media were changed after 16 hours with complete MEM. After an additional 24 hours, the cells were collected and lysed by three freeze-thaw cycles. Viral supernatants were generated by centrifugation at 10,000 g for 5 minutes and further purified by CsCl-gradient ultracentrifugation; the titer for each rAAV were determined by dot blot assay. This assay provides a titer of total number of particles per unit volume. The supernatant containing rAAV were stored in aliquots at −80 C and thawed for use immediately before each experiment.

X-gal In Situ Staining.

Samples were fixed in 0.2% gluteraldehyde and 3% paraformaldehyde for 5 minutes, and washed twice with PBS. The samples were immersed in a staining solution containing 100 mM sodium phosphate (pH 7.3), 1.3 mM MgCl₂, 3 mM K₃Fe(CN)₆, 3 mM K₄Fe(CN)₆, and 5-bromo-4-chloro-3-indolyl-3-D-galactoside (X-gal, 1 mg/ml) and incubated at 37° C. for 18 hours. The stained samples are washed twice with PBS and examined.

Echocardiographic Determination of Left Ventricular Function:

Echocardiographic imaging of left ventricle dimensions was performed using a Hewlett Packard Sonos 5500 equipped with a 8-12 MHz vascular transducer. Measurements were performed at the mid-papillary level of the left ventricle in a blinded fashion. End diastolic diameter (EDD), end systolic diameter (ESD), anterior wall thickness (AWT) and posterior wall thickness (PWT) were obtained from the M-mode echocardiographic images according to the guidelines of the American Society for echocardiography leading-edge method. For each measurement, data from at least three consecutive cardiac cycles were averaged. End systolic (ESA) and end diastolic (EDA) were determined from the short axis view of the left ventricle at the papillary muscle level to evaluate LV ejection fraction (EF). Left ventricular fractional shortening (FS) and EF were calculated according to the following formulas: LV FS (%)=[(EDD−ESD)/EDD]×100; and LV EF (%)=[(EDA−ESA)/EDA]×100

Histology and Immunohistochemical Analysis.

At 24 hr after reperfusion, hearts were flushed in situ with PBS (pH 7.4) and perfused retrograde with 50 ml of 10% phosphate buffered formalin. The hearts were harvested, washed in PBS and post-fixed in 10% formalin overnight at 4° C. The specimens were processed, embedded and sectioned at a thickness of 5 μm. Immunohistochemical staining were performed.

Measurement of Heme Oxygenase Activity:

Total heme oxygenase was measured in the microsomal fraction isolated from left ventricular homogenates. Tissues were homogenized (˜3 ml per g tissue) in ice-cold homogenization buffer (30 mM Tris-HCl, pH 7.5), 0.25 M sucrose, 0.15 M NaCl) containing protease inhibitor cocktail (Sigma). The homogenates were centrifuged at 10,000 g for 15 minutes. The supernatant fraction was centrifuged at 100,000×g for 1 h. The microsomal pellet was resuspended in 50 mM potassium phosphate buffer (pH 7.4) and sonicated on ice for 5 seconds. Heme oxygenase activity was measured as the rate of appearance of bilirubin by a spectrophotometric method.

Assessment of Oxidative Stress and Oxidative Damage:

Oxidative damage was assessed by detecting oxidation-modified protein carbonyl groups in left ventricular homogenates using the OxyBlot kit (Intergen, New York, N.Y.) according to the instructions provided by the vendor, and by quantification of total lipid peroxides (malondialdehyde and 4-hydroxynoneal) using a commercially available kit (Calbiochem, Darmstadt, Germany). Immunostaining of formalin-fixed ventricular sections with polyclonal antibody MAL-2 (anti-malondialdehyde-lysine; donated by J. Witztum, La Jolla, Calif.) were used for in situ detection of oxidation-specific lipid-protein adducts as described previously (Melo et al, 2002). The integrated density of all bands corresponding to modified proteins in each lane was used for quantification of protein oxidation using a flatbed scanner and NIH Image 1.52 program.

Determination of Apoptosis:

Apoptosis was determined by detection of inter-nucleosomal fragmentation of genomic DNA using the Apoptotic DNA ladder kit (Roche, Indianapolis, Ind.), and by terminal deoxynucleotide transferase-mediated dUTP nick end-labeling (TUNEL) in paraffin-embedded sections, using the In Situ Cell Death detection anti fluorescein-dUTP peroxidase kit (Roche, Indianapolis, Ind.). For quantification of apoptosis by DNA laddering, genomic DNA were labeled with ³²P-dUTP (NEN, Cambridge, Mass.) using terminal deoxynucleotidyl transferase (Roche, Indianapolis, Ind.) for 1 hr at 37° C. The gel were exposed to Hyperfilm for 72 hr at −80° C. with intensifying screens. The integrated density of all the bands in the lane were used for quantification of apoptosis.

Animal Surgery:

In preparation for surgery, the animals were lightly anesthetized initially by inhalation of 20% halothane:80 mineral oil mixture. Anesthesia were induced by intraperitoneal injection of a mixture of ketamine:xylazine (150:200 mg/kg BW) in sterile 0.9% NaCl and maintained with supplemental doses of the anesthetic mixture, as required. The animals were laid down in the supine position in an operating board and intubated with a blunt 17-gauge needle connected to a Harvard small rodent ventilator (Harvard Instruments, South Natick, Mass.). Tidal volume and ventilation rate were set at 2.5 ml and 60/mm, respectively during all open chest procedures. For continuous experiments, the animals were allowed to recover in their cage under a 100 W heat lamp for at least three hours prior to being returned to the animal housing premises. The animals were monitored post-operatively for 24-48 hours and administered buprenorphine (0.2 mg/kg) at 18 hr intervals if deemed to be in distress.

Statistical Analysis

All results were expressed as means±SE. One-way ANOVA coupled to Bonferroni multiple comparison test was used to compare differences between groups. P<0.05 was considered to indicate statistically significant difference.

Morphometric Determination of Infarct Size

Twenty four hours after reperfusion, the LAD was re-ligated and 0.3-0.4 ml of 1% Evans Blue in PBS (pH 7.4) were retrogradely injected into the heart via the catheter to delineate the non-ischemic area. The heart was excised and rinsed in ice cold PBS. Atrial tissue and large vessels were removed and 5-6 biventricular sections of similar thickness were made perpendicular to the long axis of the heart. The sections were incubated in 1% triphenyl tetrazolium chloride (TTC, Sigma Chemicals) in PBS (pH 7.4) for 15 min at 37° C. and photographed on both sides. The slides were projected at approximately 10 fold magnification and traced on Quad 10 to 1″ graph paper. Area at risk and infarct area were delineated and calculated for both sides of the section. The cumulative areas for all sections for each heart were used for comparisons. Infarct size was expressed as the ratio of infarct area to area at risk.

### Example 4

### Regulatable Gene Expression Using Hypoxic Response Element Constructs In Vitro

In order to develop regulatable expression of the therapeutic gene induced by specific pathophysiological stimuli several hypoxia inducible vectors were constructed and tested the efficiency of these vectors to induce gene expression during in vitro hypoxia. These vectors contain multiple tandem repeats of hypoxia responsive elements from the erythropoietin gene (Epo HREs), which were placed upstream of minimal CMV promoter followed by the luciferase gene. In addition, control vectors containing full length and minimal CMV promoter alone were constructed.

To test the efficacy of the hypoxia response elements to induce hypoxia mediated gene expression, HEK 293 cells were transfected with the following vectors: pGL3-4EpoHRE-mCMV-luc, pGL3-mCMV-luc and pGL3-fCMV-luc. Under basal conditions, cells transfected with the pGL3-fCMV vector exhibited a 10 fold higher level of expression as measured by luciferase activity when compared to cells transfected with vector containing mCMV promoter. However under hypoxic conditions, cells transfected with pGL3-mCMV-4Epo-HRE showed a 10 fold greater induction in luciferase activity as measured by relative light units (RLUs). In contrast, neither the pGL3-fCMV nor the vector containing mCMV alone responded to hypoxia.

These results indicate that pGL3-4EpoHRE construct containing a minimal CMV promoter results in low basal levels of gene expression that was then induced 10 fold under hypoxic conditions. On the contrary a vector containing just the mCMV promoter without HREs gave a very low basal and did not result in the induction of luciferase activity. We have also constructed the AAV vectors with up to five tandem repeats of hypoxia response elements from the erythropoietin gene, the minimal CMV promoter and GFP as the reporter gene and tested their efficiency to induce GFP expression under hypoxia The preliminary result showed that 5xEpoHRE resulted in further increased GFP expression in response to hypoxia.

### Example 5

### Identification of Differential Gene Expression in Cardiac Disorders

The molecular mechanisms underpining acute myocardial repair were investigated using a murine model of an acute cardiac disorder, myocardial ischemia. Murine myocardial infarctions were created by permanent ligation of left anterior descending arteries and tissues including the infarcted zone and bordering region were isolated after 1, 8 or 24 hours; cardiac tissue from sham-operated littermates served as controls. RNA was extracted from the infarcted and bordering regions and analyzed on AFFYMETRIX™ Mouse Set 430 microarrays. Reverse-transcription PCR (RT-PCR) was used to verify differentially expressed genes. A subset of 462 genes related to cell adhesion, chemokines, cytokines and chemotaxis was identified. Table 1 lists significantly upregulated genes in injured heart tissue compared to normal uninjured heart tissue. Table 2 lists down-regulated genes in injured heart tissue compared to normal heart tissue. Tables 4 and 5 list genes that are differentially expressed in injured heart tissue at 8 hours and 24 hours, respectively.

From 1 hour post infarction, the number of genes differentially expressed between hearts of MI and sham animals increased progressively. A significant increase in expression of several chemokines, cytokines, and cell adhesion molecules was seen at 24 hours post-injury. Upregulated genes included stromal derived factor-1 (SDF1), vascular cell adhesion molecule-1 (VCAM1), and fibronectin-1 (FN1). These ligands are important for stem cell trafficking through interactions with their receptors on BMSC.

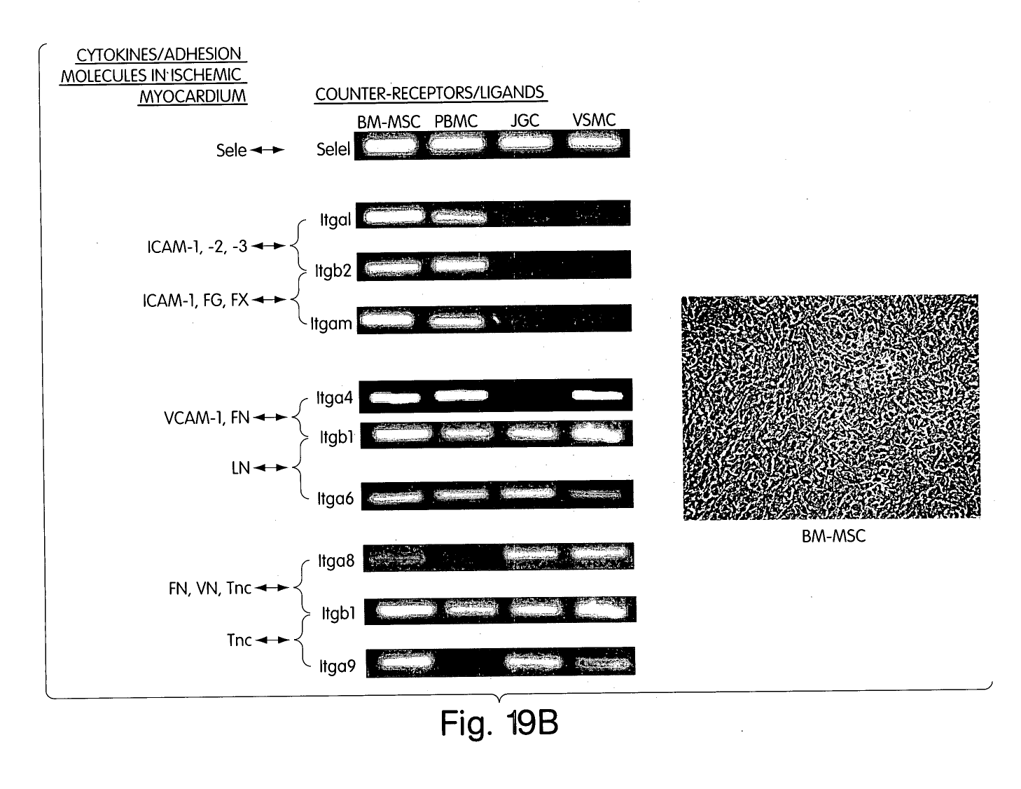

The levels of expression of the corresponding receptors to SDF1, VCAM1, FN1, IL-6, CCL2/CCL7/CCL8/CCL13, and ICAM-1 in BMSC was analyzed. Murine BMSC were isolated and cultured for 3-6 passages. RNA was isolated and analyzed by RT-PCR for the expression of receptors corresponding to the ligands. CXCR4 (for SDF 1) and integrin alpha4beta1 (for VCAM1 & FN1) are expressed in BMSC, as shown in FIGS. 19A-B. These ligand-receptor interactions (Table 3) play an important role in cardiac repair by influencing homing and migration of BMSC.

### Example 6

### Diagnosis, Prognosis and Screening Methods

The level of expression of one or more of the differentially expressed genes is determined directly from a patient derived sample, using routine methods such as PCR, Northern blotting, or chip arrays. Alternatively, the polypeptides encoded by the diffentially expressed genes are measured. Polypeptides are measured using immunospecific antibodies. The patient derived sample can be tissue isolated from the patient (e.g., cardiac tissue from a biopsy), or bodily fluids, such as blood, serum, or plasma. Alternatively, the levels of genes and/or polypeptides of interest are measured in situ.

The present invention is also useful to screen therapeutic agents that modulate the onset or progression of a cardiac disorder in a mammal. As used herein, “modulate” includes preventing or inhibiting the onset and/or progression of the cardiac disorder, as well as alleviating one or more symptoms of the cardiac disorder. The candidate agents are screened by contacting the subject with a candidate agent, determining a test level of one or more of the genes listed in Tables 1-5 in a sample derived from the subject following the contacting, and comparing the test level with a reference level of the gene. The reference level is determined by measuring the level of the gene of interest in a sample derived from a subject that does not have the cardiac disorder. An increase or decrease of the test level relative to the reference level indicates that the test agent modulates the onset or progression of the cardiac disorder. Alternatively, the level of the polypeptide encoded by a gene is determined in the subject, and compared to a reference level of the polypeptide.

TABLE 1
Up-regulated

| | |
|---|---|
| actin, beta, cytoplasmic | Actb |
| a disintegrin-like and metalloprotease | Adamts1 |
| Chemokine (C-C motif) ligand 2 | Ccl2 |
| Chemokine (C-C motif) ligand 6 | Ccl6 |
| chemokine (C-C motif) ligand 7 | Ccl7 |
| chemokine (C-C motif) ligand 9 | Ccl9 |
| chemokine (C-C motif) receptor 1 | Ccr1 |
| chemokine (C-C) receptor 2 | Ccr2 |
| procollagen, type I, alpha 1 | Col1a1 |
| chemokine (C-X-C motif) ligand 1 | Cxcl1 |
| chemokine (C-X-C motif) ligand 2 | Cxcl2 |
| chemokine (C-X-C motif) receptor 6 | Cxcr6 |
| fibronectin 1 | Fn1 |
| intercellular adhesion molecule | Icam1 |
| IFN-related developmntl regulator 1 | Ifrd1 |
| interleukin 1 receptor, type II | Il1r2 |
| interleukin 1 receptor antagonist | Il1rn |
| interleukin 6 | Il6 |
| integrin alpha 5 | Itga5 |
| integrin alpha 6 | Itga6 |
| macrophage migration inhibitory factor | Mif |
| matrix metalloproteinase 14 | Mmp14 |
| matrix metalloproteinase 8 | Mmp8 |
| NFKB It chn gene enhncr in B-cells inhibtr | Nfkbia |
| platelet factor 4 | Pf4 |
| plasminogen activator, tissue | Plat |
| urokinase plasminogen activator receptor | Plaur |
| pro-platelet basic protein | Ppbp |
| ribosomal protein L13a | Rpl13a |
| selectin, endothelial cell | Sele |
| secreted acidic cysteine rich glycoprotein | Sparc |
| transforming growth factor, beta 1 | Tgfb1 |
| transforming growth factor, beta 2 | Tgfb2 |
| thrombospondin 1 | Thbs1 |
| tissue inhibitor of metalloproteinase 1 | Timp1 |
| tenascin C | Tnc |
| vascular cell adhesion molecule 1 | Vcam1 |
| vascular endothelial growth factor A | Vegfa |

TABLE 2
Down-regulated significantly

| | |
|---|---|
| catenin alpha-like 1 | Catnal1 |
| cystatin C | Cst3 |
| interleukin 10 receptor, beta | II10rb |
| kit ligand | Kitl |
| matrix metalloproteinase 2 | Mmp2 |
| tissue inhibitor of metalloproteinase 2 | Timp2 |
| transcription factor 4 | Tcf4 |
| vitronectin | Vtn |

TABLE 3
Receptor Ligand

| Up-regulated in ischemic heart | Expressed by BM-MSC |
|---|---|
| SDF-1 | CXR4 |
| IL-6 | IL-6RA, IL-6ST |
| CCL7 | CCR2 |
| Sele | Sele |
| ICAM-1 | Itgal/b2; Itgam/b2 |
| VCAM-1 | Itga4/b1 |
| FN | Itga4/b1; Itga8/b1 |
| LN | Itga6/b1 |
| Tnc | Itga/b1, Itga9/b1 |

AT 8 Hrs

Up-regulated
significantly

| | | | |
|---|---|---|---|
| a disintegrin-like and | Adamts1 | interleukin 1 receptor, | II1r2 |
| metalloprotease | | type II | |
| actin, beta, cytoplasmic | Actb | interleukin 6 | II6 |
| chemokine (C-C motif) | Ccl2 | matrix | Mmp8 |
| ligand 2 | | metalloproteinase 8 | |
| chemokine (C-X-C | Cxcl1 | NFKB inhibitor, alpha | Nfkbia |
| motif) ligand 1 | | | |
| chemokine (C-X-C | Cxcl2 | plasminogen activator, | Plat |
| motif) ligand 2 | | tissue | |
| chemokine orphan | Cmkor1 | selectin, endothelial | Sele |
| receptor 1 | | cell | |
| integrin alpha 5 | Itga5 | thrombospondin 1 | Thbs1 |
| integrin alpha 6 | Itga6 | transforming growth | Tgfb2 |
| | | factor, beta 2 | |
| intercellular adhesion | Icam1 | vascular cell adhesion | Vcam1 |
| molecule | | molecule 1 | |
| IFN-related develop- | Ifrd1 | vascular endothelial | Vegf2 |
| mental regulator 1 | | growth factor A | |
| Down-regulated | | | |
| significantly | | | |
| interleukin 10 receptor, | Il10rb | stromal cell derived | Sdf2 |
| beta | | factor 2 | |

TABLE 5
AT 24 Hrs
Up-regulated significantly

| | |
|---|---|
| a disintegrin-like and metalloprotease | Adamts1 |
| actin, beta, cytoplasmic | Actb |
| chemokine (C-C motif) ligand 2 | Ccl2 |
| chemokine (C-C motif) ligand 6 | Ccl6 |
| chemokine (C-C motif) ligand 7 | Ccl7 |
| chemokine (C-C motif) ligand 9 | Ccl9 |
| chemokine (C-C motif) receptor 1 | Ccr1 |
| chemokine (C-C) receptor 2 | Ccr2 |
| chemokine (C-X-C motif) ligand 1 | Cxcl1 |
| chemokine (C-X-C motif) ligand 2 | Cxcl2 |
| chemokine (C-X-C motif) receptor 6 | Cxcr6 |
| fibronectin 1 | Fn1 |
| integrin alpha 5 | Itga5 |
| intercellular adhesion molecule | Icam1 |
| IFN-related developmental regulator 1 | Ifrd1 |
| interleukin 1 receptor antagonist | Il1rn |
| interleukin 1 receptor, type II | Il1r2 |
| interleukin 6 | Il6 |
| macrophage migration inhibitory factor | Mif |
| matrix metalloproteinase 14 | Mmp14 |
| NFKB inhibitor, alpha | Nfkbia |
| platelet factor 4 | Pf4 |
| procollagen, type I, alpha 1 | Col1a1 |
| pro-platelet basic protein | Ppbp |
| ribosomal protein L13a | Rpl13a |
| secreted acidic cysteine rich glycoprotein | Sparc |
| tenascin C | Tnc |
| thrombospondin 1 | Thbs1 |
| tissue inhibitor of metalloproteinase 1 | Timp1 |
| transforming growth factor, beta 1 | Tgfb1 |
| transforming growth factor, beta 2 | Tgfb2 |
| urokinase plasminogen activator receptor | Plaur |

Down-regulated significantly

| | |
|---|---|
| catenin alpha-like 1 | Catnal1 |
| cystatin C | Cst3 |
| interleukin 10 receptor, beta | Il10rb |
| kit ligand | Kitl |
| matrix metalloproteinase 2 | Mmp2 |
| tissue inhibitor of metalloproteinase 2 | Timp2 |
| transcription factor 4 | Tcf4 |
| vitronectin | Vtn |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. A method of regenerating a mesenchymally-derived tissue, comprising contacting said tissue with a composition comprising an isolated adult mesenchymal stem cell, said mesenchymal stem cell comprising an exogenous nucleic acid encoding an akt gene.

2. The method of claim 1, wherein said tissue is selected from the group consisting of connective tissue, epithelial tissue, nervous tissue and muscle tissue.

3. The method of claim 1, wherein said tissue is selected from the group consisting of myocardial, brain, spinal cord, bone, cartilage, liver, muscle, lung, vascular, and adipose tissue.

4. The method of claim 2, wherein said muscle tissue comprises skeletal muscle.

5. The method of claim 2, wherein said muscle tissue comprises smooth muscle.

6. The method of claim 1, wherein said mesenchymal stem cell further comprises an exogenous nucleic acid encoding a homing molecule.

7. The method of claim 6, wherien said homing molecule is selected from the group consisting of a chemokine receptor, an interleukin receptor, an estrogen receptor, an integrin receptor

8. The method of claim 1, wherein said mesenchymal stem cell further comprises an exogenous nucleic acid encoding a hormone.

9. The method of claim 1, wherein said mesenchymal stem cell further comprises an exogenous nucleic acid encoding an angiogenic factor.

10. The method of claim 1, wherein said mesenchymal stem cell further comprises an exogenous nucleic acid encoding a bone morphogenetic protein.

11. The method of claim 1, wherein said mesenchymal stem cell further comprises an exogenogenous nucleic acid encoding an extracellular matrix protein.

12. The method of claim 1, wherein said mesenchymal stem cell further comprises an exogenous nucleic acid encoding a cytokine or growth factor.

13. A composition comprising an apoptosis-resistant primary stem cell, said stem cell comprising an exogenous akt gene, wherein apoptosis of said cell is reduced by at least 10% compared to a primary mesenchymal stem cell lacking said akt gene.

14. The composition of claim 13, wherein said stem cell is an adult bone-marrow derived mesenchymal cell.

15. The composition of claim 13, wherein said apoptosis is reduced by at least 50%.

16. The composition of claim 13, wherein said apoptosis is reduced by at least 2-fold.

17. The composition of claim 13, wherein said apoptosis is reduced by at least 5-fold.

18. The composition of claim 13, wherein said apoptosis is reduced by at least 10-fold.

19. The composition of claim 13, wherein said stem cell is non-tumor forming.

20. The composition of claim 13, wherein said stem cell further comprises a homing receptor.

21. A method of regenerating an injured myocardial tissue, said method comprising contacting said tissue with the composition comprising an isolated adult recombinant mesenchymal stem cell (rMSC), said rMSC comprising an exogenous nucleic acid operably linked to a promoter, wherein said nucleic acid expresses a therapeutically effective amount of an anti-apoptotic gene.

22. The method of claim 21 wherein said mesenchymal stem cells are administered to an individual to regenerate or repair cardiac muscle that has been damaged through disease.

23. The method of claim 22 wherein said mesenchymal stem cells are administered to an individual who has suffered myocardial infarction.

24. The method of claim 22 wherein said mesenchymal stem cells are administered directly to the heart.

25. The method of claim 22 wherein said mesenchymal stem cells are administered systemically.

26. The method of claim 25 wherein said mesenchymal stem cells are administered by injection.

27. The method of claim 22 wherein said mesenchymal stem cells are human.

28. The method of claim 27 wherein said mesenchymal stem cells are administered to an individual who has suffered myocardial infarction.

29. The method of claim 28 wherein said mesenchymal stem cells are administered directly to the heart.

30. The method of claim 28 wherein said the mesenchymal stem cells are administered systemically.

31. A method of regenerating myocardial tissue, said method comprising contacting said tissue with the composition comprising an isolated adult recombinant mesenchymal stem cell (rMSC), said rMSC comprising an exogenous nucleic acid operably linked to a promoter, wherein said nucleic acid expresses a therapeutically effective amount of a cell protective polypeptide, wherein the expression of said polypeptide is induced by a triggering agent or condition.

32. The method of claim 31, wherein the condition is hypoxia or oxidative stress.

33. The method of claim 31, wherein the agent is an antibiotic

34. The method of claim 33, wherein said antibiotic is tetracycline.

35. The method of claim 31, wherein the agent is an immunosuppressive.

36. The method of claim 35, wherein said immunosuppressive is rapamycin.

37. The method of claim 31, wherein the agent is a hormone receptor antagonist.

38. The method of claim 37, wherein said hormone receptor antagonist is mifepristone.

39. The method of claim 31, wherein the cell protective polypeptide is selected from the group consisting of an antioxidant enzyme protein, a heat shock protein, an anti-inflammatory protein, a survival protein, an anti-apoptotic protein, a coronary vessel tone protein, a pro-angiogenic protein, a contractility protein, a plaque stabilization protein, a thromboprotection protein, a blood pressure protein and a vascular cell proliferation protein.

40. The method of claim 31, wherein the subject is at risk of developing a condition characterized by aberrant cell damage.

41. The method of claim 31, wherein said aberrant cell damage is apoptotic cell death.

42. The method of claim 31, wherein said subject is at risk of developing stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy.

43. The method of claim 21, wherein at least 20% of said injured myocardial tissue is regenerated.

44. A composition comprising an isolated mesenchymal stem cell comprising an exogenous nucleic acid encoding a tissue protective polypeptide, a oxygen sensitive regulatory element and a cell targeting element, wherein the expression of said polypeptide is regulated by said regulatory element

45. The composition of claim 44, wherein said oxygen sensitive regulatory element is a hypoxia response element.

46. The composition of claim 44, wherein said oxygen sensitive regulatory element is a oxidative stress response element.

47. The composition of claim 46, wherein said oxidative stress response element is a peroxidase promoter.

48. The composition of claim 44, wherein said cell targeting element is selected from the group consisting of α-MHC, myosin light chain-2, troponin T.

49. The composition of claim 44, wherein the composition comprises vector selected from the group consisting of an adeno-associated virus vector, lentivirus vector retrovirus vector.

50. The composition of claim 44, wherein the composition comprises an adeno-associated virus vector.

51. A method of inhibiting apoptosis of engrafted cells in a mammal, said method comprising administering to said mammal a composition comprising an isolated mesenchymal stem cell comprising an exogenous nucleic acid encoding a polypeptide selected from the group consisting of: an extracellular superoxide dismutase polypeptide, a heme oxygenase polypeptide, and an Akt polypeptide, wherein said mammal is suffering from or at risk of developing a cardiac disorder.

52. The method of claim 51, wherein said cardiac disorder is selected from the group consisting of chronic coronary ischemia, arteriosclerosis, congestive heart failure, angina, atherosclerosis, and myocardial hypertrophy.

53. The method of claim 51, wherein said composition comprises an adeno-associated virus vector.

54. The method of claim 51, wherein the human heme oxygenase-1 nucleic acid is operatively linked to a promoter.

55. The method of claim 54, wherein said promoter is a human cytomegalovirus immediate early promoter.

56. The method of claim 51, wherein said human heme oxygenase-1 nucleic acid is operatively linked to a bovine growth hormone polyadenylation signal.

57. The method of claim 56, wherein said bovine growth hormone polyadenylation signal is flanked by the adeno-associated viral inverted terminal repeats.

58. The method of claim 51, wherein said composition is administered at a dose sufficient to increase survival of engrafted mesenchymal stem cell oxidative stress-induced cardiomyocyte cell death.

59. A method of increasing post-transplantation survival of engrafted cells in a mammal in a mammal, said method comprising administering to said mammal a composition comprising an isolated mesenchymal stem cell comprising an exogenous nucleic acid encoding a polypeptide selected from the group consisting of: an extracellular superoxide dismutase polypeptide, a heme oxygenase polypeptide, and an Akt polypeptide, thereby increasing survival of the engrafted cells.

60. The method of claim 59, mammal is at risk of a cardiac disorder.

61. The method of claim 59, wherein said cardiac disorder is selected from the group consisting of myocardial infarction, chronic coronary ischemia, arteriosclerosis, congestive heart failure, angina, atherosclerosis, and myocardial hypertrophy.

62. The method of claim 59, wherein said composition comprises an adeno-associated virus vector.

63. The method of claim 59, wherein the extracellular superoxide dismutase nucleic acid is operatively linked to a promoter.

64. The method of claim 63, wherein said promoter is a human cytomegalovirus immediate early promoter.

65. The method of claim 59, wherein said extracellular superoxide dismutase polypeptide nucleic acid is operatively linked to a bovine growth hormone polyadenylation signal.

66. The method of claim 65, wherein said bovine growth hormone polyadenylation signal is flanked by the adeno-associated viral inverted terminal repeats.

67. The method of claim 59, wherein said composition is administered at a dose sufficient to inhibit oxidative stress-induced cardiomyocyte cell death.

68. A method of treating a cardiac disorder, comprising identifying a mammal suffering from or at risk of developing said disorder and administering to said mammal a composition comprising an isolated mesenchymal stem cell comprising an exogenous nucleic acid expressing therapeutic amounts of a polypeptide selected from the group consisting of: an extracellular superoxide dismutase polypeptide, a heme oxygenase polypeptide, and an Akt polypeptide.

69. The method of claim 68, wherein said composition comprises an adeno-associated virus vector.

70. The method of claim 69, wherein the extracellular superoxide dismutase polypeptide nucleic acid is operatively linked to a promoter.

71. The method of claim 70, wherein said promoter is a human cytomegalovirus immediate early promoter.

72. The method of claim 68, wherein said extracellular superoxide dismutase nucleic acid is operatively linked to a bovine growth hormone polyadenylation signal.

73. The method of claim 72, wherein said bovine growth hormone polyadenylation signal is flanked by the adeno-associated viral inverted terminal repeats.

74. The method of claim 68, wherein said cardiac disorder is selected from the group consisting of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy, and stroke.

75. A cardioprotective agent comprising a recombinant adeno-associated viral vector comprising nucleotide encoding a extracellular superoxide dismutase polypeptide operatively linked to a human cytomegalovirus immediate early promoter.

76. The cardioprotective agent of claim 75, further comprising a bovine growth hormone polyadenylation signal.

77. The cardioprotective agent of claim 76, wherein said bovine growth hormone polyadenylation signal is flanked by the adeno-associated viral inverted terminal repeats.

78. A method for reducing scar formation in infarcted heart tissue, said method comprising contacting said tissue with the composition comprising an isolated adult recombinant mesenchymal stem cell (rMSC), said rMSC comprising an exogenous nucleic acid operably linked to a promoter, wherein said nucleic acid expresses a therapeutically effective amount of an anti-apoptotic gene.

79. A composition comprising an isolated adult recombinant mesenchymal stem cell (rMSC), said rMSC comprising an exogenous nucleic acid operably linked to a promoter, wherein said nucleic acid expresses a therapeutically effective amount of an anti-apoptotic gene.

80. The composition of claim 79, wherein the anti-apoptotic gene is selected from the group consisting of an Akt gene, an extracellular superoxide dismutase (ecSOD) polypeptide and a heme oxygenase gene.

81. A method of enhancing migration of a stem cell to an injured tissue, comprising increasing the amount of a stem cell polypeptide on the surface of said stem cell, wherein said stem cell polypeptide is selected from the group consisting of CXCR4, IL-6RA, IL-6ST, CCR2, Selel, Itgal/b2, Itgam/b2, Itga41b 1, Itga8/b 1, Itga6/b 1, and Itga9/b 1.

82. The method of claim 81, wherein said cell is a bone marrow-derived stem cell.

83. The method of claim 81, wherein said cell is a mesenchymal stem cell.

84. The method of claim 81, wherein said method comprises introducing into said stem cell a nucleic acid encoding said receptor.

85. A method of enhancing engraftment of a stem cell to an injured tissue, comprising increasing the amount of an injury-associated polypeptide in said injured tissue, wherein said injury-associated polyeptide is selected from the group consisting of SDF1, IL-6, CCL2, Sele, ICAM-1, VCAM-1, FN, LN, and Tnc.

86. The method of claim 85, wherein said injured tissue is cardiac tissue.

87. The method of 85, wherein said injured tissue is ischemic myocardial tissue.

88. The method of claim 85, wherein said method comprises contacting said injured tissue with a nucleic acid encoding said injury-associated polypeptide.

89. The method of claim 85, wherein said method comprises contacting said injured tissue with said injury-associated polypeptide.

90. The method of claim 85, wherein said method comprises injecting said injury-associated polypeptide or a nucleic acid encoding said polypeptide directly into the myocardium.


SEQUENCE LISTING (2 sequences)

SEQ ID NO: 1 - DNA, 30 bp, Artificial sequence
  oligonucleotide primer
gcaagatctgataccatgaacgacgtagcc

SEQ ID NO: 2 - DNA, 27 bp, Artificial sequence
  Oligonucleotide primer
cggtcaccgtgtcggactcctaggatc